SEQUENCE LISTING (13 sequences)

SEQ ID NO: 1 - protein, 758 aa, synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMESGGGSGGGSGGGSGGGKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 2 - protein, 450 aa, synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALME

SEQ ID NO: 3 - protein, 292 aa, synthetic construct
KPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 4 - protein, 16 aa, synthetic construct
SGGGSGGGSGGGSGGG

SEQ ID NO: 5 - DNA, 2306 bp, synthetic construct
  source 1..2306: mol_type = other DNA
gctagcactagtaccatggcgaaaccgaccagcaaagatagcggcctgaaagaaaaatttaaaattctgctgggcctgggcaccccgcgcccgaacccgcgcagcgcggaaggcaaacagaccgaatttattattaccgcggaaattctgcgcgaactgagcatggaatgcggcctgaacaaccgcattcgcatgattggccagatttgcgaagtggcgaaaaccaaaaaatttgaagaacatgcggtggaagcgctgtggaaagcggtggcggatctgctgcagccggaacgcccgctggaagcgcgccatgcggtgctggcgctgctgaaagcgattgtgcagggccagggcgaacgcctgggcgtgctgcgcgcgctgttttttaaagtgattaaagattatccgagcaacgaagatctgcatgaacgcctggaagtgtttaaagcgctgaccgataacggccgccatattacctatctggaagaagaactggcggattttgtgctgcagtggatggatgtgggcctgagcagcgaatttctgctggtgctggtgaacctggtgaaatttaacagctgctatctggatgaatatattgcgcgcatggtgcagatgatttgcctgctgtgcgtgcgcaccgcgagcagcgtggatattgaagtgagcctgcaggtgctggatgcggtggtgtgctataactgcctgccggcggaaagcctgccgctgtttattgtgaccctgtgccgcaccattaacgtgaaagaactgtgcgaaccgtgctggaaactgatgcgcaacctgctgggcacccatctgggccatagcgcgatttataacatgtgccatctgatggaagatcgcgcgtatatggaagatgcgccgctgctgcgcggcgcggtgttttttgtgggcatggcgctgtggggcgcgcatcgcctgtatagcctgcgcaacagcccgaccagcgtgctgccgagcttttatcaggcgatggcgtgcccgaacgaagtggtgagctatgaaattgtgctgagcattacccgcctgattaaaaaatatcgcaaagaactgcaggtggtggcgtgggatattctgctgaacattattgaacgcctgctgcagcagctgcagaccctggatagcccggaactgcgcaccattgtgcatgatctgctgaccaccgtggaagaactgtgcgatcagaacgaatttcatggcagccaggaacgctattttgaactggtggaacgctgcgcggatcagcgcccggaaagcagcctgctgaacctgattagctatcgcgcgcagagcattcatccggcgaaagatggctggattcagaacctgcaggcgctgatggaatctggtgggggtagcggaggagggtcagggggcggcagtggaggcggaaaaccgattctgctgccgaacgaaagccagagctttgaacgcagcgtgcagctgctggatcagattccgagctatgatacccataaaattgcggtgctgtatgtgggcgaaggccagagcaacagcgaactggcgattctgagcaacgaacatggcagctatcgctataccgaatttctgaccggcctgggccgcctgattgaactgaaagattgccagccggataaagtgtatctgggcggcctggatgtgtgcggcgaagatggccagtttacctattgctggcatgatgatattatgcaggcggtgtttcatattgcgaccctgatgccgaccaaagatgtggataaacatcgctgcgataaaaaacgccatctgggcaacgattttgtgagcattgtgtataacgatagcggcgaagattttaaactgggcaccattaaaggccagtttaactttgtgcatgtgattgtgaccccgctggattatgaatgcaacctggtgagcctgcagtgccgcaaagatatggaaggcctggtggataccagcgtggcgaaaattgtgagcgatcgcaacctgccgtttgtggcgcgccagatggcgctgcatgcgaacatggcgagccaggtgcatcatagccgcagcaacccgaccgatatttatccgagcaaatggattgcgcgcctgcgccatattaaacgcctgcgccagcgcatttgcgaagaagcggcgtatagcaacccgagcctgccgctggtgcatccgccgagccatagcaaagcgccggcgcagaccccggcggaaccgaccccgggctatgaagtgggccagcgcaaacgcctgattagcagcgtggaagattttaccgaatttgtgtaggcggccgcctcgag

SEQ ID NO: 6 - DNA, 1353 bp, synthetic construct
  source 1..1353: mol_type = other DNA
accatggcgaaaccgaccagcaaagatagcggcctgaaagaaaaatttaaaattctgctgggcctgggcaccccgcgcccgaacccgcgcagcgcggaaggcaaacagaccgaatttattattaccgcggaaattctgcgcgaactgagcatggaatgcggcctgaacaaccgcattcgcatgattggccagatttgcgaagtggcgaaaaccaaaaaatttgaagaacatgcggtggaagcgctgtggaaagcggtggcggatctgctgcagccggaacgcccgctggaagcgcgccatgcggtgctggcgctgctgaaagcgattgtgcagggccagggcgaacgcctgggcgtgctgcgcgcgctgttttttaaagtgattaaagattatccgagcaacgaagatctgcatgaacgcctggaagtgtttaaagcgctgaccgataacggccgccatattacctatctggaagaagaactggcggattttgtgctgcagtggatggatgtgggcctgagcagcgaatttctgctggtgctggtgaacctggtgaaatttaacagctgctatctggatgaatatattgcgcgcatggtgcagatgatttgcctgctgtgcgtgcgcaccgcgagcagcgtggatattgaagtgagcctgcaggtgctggatgcggtggtgtgctataactgcctgccggcggaaagcctgccgctgtttattgtgaccctgtgccgcaccattaacgtgaaagaactgtgcgaaccgtgctggaaactgatgcgcaacctgctgggcacccatctgggccatagcgcgatttataacatgtgccatctgatggaagatcgcgcgtatatggaagatgcgccgctgctgcgcggcgcggtgttttttgtgggcatggcgctgtggggcgcgcatcgcctgtatagcctgcgcaacagcccgaccagcgtgctgccgagcttttatcaggcgatggcgtgcccgaacgaagtggtgagctatgaaattgtgctgagcattacccgcctgattaaaaaatatcgcaaagaactgcaggtggtggcgtgggatattctgctgaacattattgaacgcctgctgcagcagctgcagaccctggatagcccggaactgcgcaccattgtgcatgatctgctgaccaccgtggaagaactgtgcgatcagaacgaatttcatggcagccaggaacgctattttgaactggtggaacgctgcgcggatcagcgcccggaaagcagcctgctgaacctgattagctatcgcgcgcagagcattcatccggcgaaagatggctggattcagaacctgcaggcgctgatggaa

SEQ ID NO: 7 - DNA, 876 bp, synthetic construct
  source 1..876: mol_type = other DNA
aaaccgattctgctgccgaacgaaagccagagctttgaacgcagcgtgcagctgctggatcagattccgagctatgatacccataaaattgcggtgctgtatgtgggcgaaggccagagcaacagcgaactggcgattctgagcaacgaacatggcagctatcgctataccgaatttctgaccggcctgggccgcctgattgaactgaaagattgccagccggataaagtgtatctgggcggcctggatgtgtgcggcgaagatggccagtttacctattgctggcatgatgatattatgcaggcggtgtttcatattgcgaccctgatgccgaccaaagatgtggataaacatcgctgcgataaaaaacgccatctgggcaacgattttgtgagcattgtgtataacgatagcggcgaagattttaaactgggcaccattaaaggccagtttaactttgtgcatgtgattgtgaccccgctggattatgaatgcaacctggtgagcctgcagtgccgcaaagatatggaaggcctggtggataccagcgtggcgaaaattgtgagcgatcgcaacctgccgtttgtggcgcgccagatggcgctgcatgcgaacatggcgagccaggtgcatcatagccgcagcaacccgaccgatatttatccgagcaaatggattgcgcgcctgcgccatattaaacgcctgcgccagcgcatttgcgaagaagcggcgtatagcaacccgagcctgccgctggtgcatccgccgagccatagcaaagcgccggcgcagaccccggcggaaccgaccccgggctatgaagtgggccagcgcaaacgcctgattagcagcgtggaagattttaccgaatttgtg

SEQ ID NO: 8 - DNA, 48 bp, synthetic construct
  source 1..48: mol_type = other DNA
tctggtgggggtagcggaggagggtcagggggcggcagtggaggcgga

SEQ ID NO: 9 - DNA, 7540 bp, synthetic construct
  source 1..7540: mol_type = other DNA
ggccgctctagaagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggactagagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaaggggttcaagcttaaaaactagtaccatggcaaaacctacatcaaaagatagcggactgaaagagaagttcaaaatcctgctgggactggggacaccacgccctaatccacggtccgccgagggcaagcagaccgagttcatcatcacagccgagatcctgcgcgagctgtctatggagtgcggcctgaacaatcggatcagaatgatcggccagatttgcgaggtggccaagaccaagaagtttgaggagcacgcagtggaggccctgtggaaggcagtggccgatctgctgcagcctgagagaccactggaggcaaggcacgccgtgctggccctgctgaaggccatcgtgcagggacagggagagcgcctgggcgtgctgcgggccctgttctttaaagtgatcaaggactaccctagcaacgaggatctgcacgagagactggaggtgttcaaggccctgaccgataatggcaggcacatcacatatctggaggaggagctggccgactttgtgctgcagtggatggatgtgggcctgagctccgagttcctgctggtgctggtgaacctggtgaagtttaattcctgctacctggacgagtatatcgcccgcatggtgcagatgatctgcctgctgtgcgtgcggaccgcctctagcgtggacatcgaggtgtctctgcaggtgctggatgccgtggtgtgctacaactgtctgcccgccgagagcctgcctctgttcatcgtgaccctgtgcagaacaatcaatgtgaaggagctgtgcgagccttgttggaagctgatgaggaacctgctgggcacacacctgggacacagcgccatctacaatatgtgccacctgatggaggaccgcgcctatatggaggatgcaccactgctgaggggagccgtgttctttgtgggaatggcactgtggggagcacacagactgtactccctgaggaactctccaaccagcgtgctgccctctttttaccaggctatggcctgtccaaatgaggtggtgtcttatgagatcgtgctgagcatcacacgcctgatcaagaagtatcggaaggagctgcaggtggtggcctgggacatcctgctgaacatcatcgagcgcctgctgcagcagctgcagaccctggacagcccagagctgaggacaatcgtgcacgatctgctgaccacagtggaggagctgtgcgaccagaatgagttccacggctcccaggagcggtactttgagctggtggagcggtgcgcagatcagaggccagagtcctctctgctgaacctgatctcctatcgggcccagtctatccaccctgccaaggacggctggattcagaatctgcaggccctgatggagagcggaggaggctccggaggaggctctggaggcggcagcggcggcggcaagccaatcctgctgcccaacgagagccagagcttcgagcggagcgtgcagctgctggaccagatccccagctacgatacccacaagatcgccgtgctgtatgtgggcgagggccagtctaacagcgagctggccatcctgagcaatgagcacggctcctacagatataccgagtttctgacaggcctgggcaggctgatcgagctgaaggactgccagcccgataaggtgtacctgggaggcctggacgtgtgcggagaggatggccagttcacctattgttggcacgacgatatcatgcaggccgtgtttcacatcgccaccctgatgcctacaaaggacgtggataagcacagatgtgacaagaagaggcacctgggcaacgatttcgtgtccatcgtgtacaatgactctggcgaggacttcaagctgggcaccatcaagggccagttcaactttgtgcacgtgatcgtgacaccactggattatgagtgcaatctggtgagcctgcagtgtagaaaggacatggagggcctggtggataccagcgtggccaagatcgtgtccgacagaaacctgcccttcgtggccaggcagatggccctgcacgccaacatggccagccaggtgcaccactccaggtctaatcctacagacatctacccatccaagtggatcgccaggctgcgccacatcaagcggctgagacagaggatctgcgaggaggccgcctattccaatccctctctgcctctggtgcacccacctagccactccaaggcacctgcacagaccccagcagagccaacaccaggatacgaagtgggacagaggaagcggctgatctcctccgtggaagatttcaccgaatttgtggagcagaagctgattagcgaagaagacctgtaagc

SEQ ID NO: 10 - protein, 1807 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 11 - protein, 768 aa, synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMESGGGSGGGSGGGSGGGKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFVEQKLISEEDL

SEQ ID NO: 12 - DNA, 2336 bp, synthetic construct
  source 1..2336: mol_type = other DNA
gctagcactagtaccatggcgaaaccgaccagcaaagatagcggcctgaaagaaaaatttaaaattctgctgggcctgggcaccccgcgcccgaacccgcgcagcgcggaaggcaaacagaccgaatttattattaccgcggaaattctgcgcgaactgagcatggaatgcggcctgaacaaccgcattcgcatgattggccagatttgcgaagtggcgaaaaccaaaaaatttgaagaacatgcggtggaagcgctgtggaaagcggtggcggatctgctgcagccggaacgcccgctggaagcgcgccatgcggtgctggcgctgctgaaagcgattgtgcagggccagggcgaacgcctgggcgtgctgcgcgcgctgttttttaaagtgattaaagattatccgagcaacgaagatctgcatgaacgcctggaagtgtttaaagcgctgaccgataacggccgccatattacctatctggaagaagaactggcggattttgtgctgcagtggatggatgtgggcctgagcagcgaatttctgctggtgctggtgaacctggtgaaatttaacagctgctatctggatgaatatattgcgcgcatggtgcagatgatttgcctgctgtgcgtgcgcaccgcgagcagcgtggatattgaagtgagcctgcaggtgctggatgcggtggtgtgctataactgcctgccggcggaaagcctgccgctgtttattgtgaccctgtgccgcaccattaacgtgaaagaactgtgcgaaccgtgctggaaactgatgcgcaacctgctgggcacccatctgggccatagcgcgatttataacatgtgccatctgatggaagatcgcgcgtatatggaagatgcgccgctgctgcgcggcgcggtgttttttgtgggcatggcgctgtggggcgcgcatcgcctgtatagcctgcgcaacagcccgaccagcgtgctgccgagcttttatcaggcgatggcgtgcccgaacgaagtggtgagctatgaaattgtgctgagcattacccgcctgattaaaaaatatcgcaaagaactgcaggtggtggcgtgggatattctgctgaacattattgaacgcctgctgcagcagctgcagaccctggatagcccggaactgcgcaccattgtgcatgatctgctgaccaccgtggaagaactgtgcgatcagaacgaatttcatggcagccaggaacgctattttgaactggtggaacgctgcgcggatcagcgcccggaaagcagcctgctgaacctgattagctatcgcgcgcagagcattcatccggcgaaagatggctggattcagaacctgcaggcgctgatggaatctggtgggggtagcggaggagggtcagggggcggcagtggaggcggaaaaccgattctgctgccgaacgaaagccagagctttgaacgcagcgtgcagctgctggatcagattccgagctatgatacccataaaattgcggtgctgtatgtgggcgaaggccagagcaacagcgaactggcgattctgagcaacgaacatggcagctatcgctataccgaatttctgaccggcctgggccgcctgattgaactgaaagattgccagccggataaagtgtatctgggcggcctggatgtgtgcggcgaagatggccagtttacctattgctggcatgatgatattatgcaggcggtgtttcatattgcgaccctgatgccgaccaaagatgtggataaacatcgctgcgataaaaaacgccatctgggcaacgattttgtgagcattgtgtataacgatagcggcgaagattttaaactgggcaccattaaaggccagtttaactttgtgcatgtgattgtgaccccgctggattatgaatgcaacctggtgagcctgcagtgccgcaaagatatggaaggcctggtggataccagcgtggcgaaaattgtgagcgatcgcaacctgccgtttgtggcgcgccagatggcgctgcatgcgaacatggcgagccaggtgcatcatagccgcagcaacccgaccgatatttatccgagcaaatggattgcgcgcctgcgccatattaaacgcctgcgccagcgcatttgcgaagaagcggcgtatagcaacccgagcctgccgctggtgcatccgccgagccatagcaaagcgccggcgcagaccccggcggaaccgaccccgggctatgaagtgggccagcgcaaacgcctgattagcagcgtggaagattttaccgaatttgtggaacaaaaactcatctcagaagaggatctgtaggcggccgcctcgag

SEQ ID NO: 13 - protein, 4 aa, synthetic construct
SGGG

(19) United States
(12) Patent Application Publication
(10) Pub. No.: US 2024/0343768 A1
(43) Pub. Date: Oct. 17, 2024

(54) GENE THERAPY FOR TUBEROUS SCLEROSIS

(71) Applicant: The General Hospital Corporation, Boston, MA (US)

(72) Inventors: Xandra BREAKEFIELD, Newton, MA (US); Casey MAGUIRE, Arlington, MA (US); Shilpa PRABHAKAR, Braintree, MA (US); David YELLEN, Newton, MA (US)

(21) Appl. No.: 18/602,622

(22) Filed: Mar. 12, 2024

Related U.S. Application Data
(63) Continuation of application No. 16/613,907, filed on Nov. 15, 2019, now Pat. No. 11,958,887.
(60) Provisional application No. 62/507,358, filed on May 17, 2017.

(51) Int. Cl. C07K 14/47; A61K 9/00; A61K 31/436; A61K 38/17; A61K 48/00; A61P 35/00; C12N 7/00; C12N 15/86

(52) U.S. Cl. CPC C07K 14/4705; A61K 9/0019; A61K 9/007; A61K 9/0085; A61K 31/436; A61K 38/1709; A61K 48/005; A61P 35/00; C12N 7/00; C12N 15/86; C12N 2750/14143; C12N 2750/14171; C12N 2830/50

(57) ABSTRACT

The invention provides compositions and methods for treating tuberous sclerosis complex (TSC). In particular, provided are condensed tuberins (cTuberins), cTuberin nucleic acids, and recombinant adeno-associated viruses (rAAVs) carrying a cTuberin nucleic acid for treating a patient with TSC.

### CROSS-REFERENCE TO RELATED APPLICATIONS

This application claims priority to U.S. Provisional Application No. 62/507,358, filed on May 17, 2017, which is hereby incorporated by reference in its entirety.

### STATEMENT AS TO FEDERALLY FUNDED RESEARCH

This invention was made with government support under Grant No. TS120038 awarded by the Department of Defense. The government has certain rights in the invention.

### SEQUENCE LISTING

The instant application contains a Sequence Listing which has been submitted electronically in ASCII format and is hereby incorporated by reference in its entirety. Said ASCII copy, created on May 15, 2018, is named 51317-002WO2_Sequence_Listing_5.15.18_ST25 and is 55,672 bytes in size.

### BACKGROUND OF THE INVENTION

Tuberous sclerosis complex (TSC) is a tumor suppressor syndrome inherited in an autosomal dominant manner with an incidence of about 1 in 5,500. Patients inherit a mutation in one allele of the TSC1 gene (encoding hamartin) or the TSC2 gene (encoding tuberin). These proteins together suppress mammalian target of rapamycin complex 1 (mTORC1) activity. If a mutation in the corresponding normal allele occurs during development or in some somatic cells, it results in enlargement and increased proliferation of cells, forming benign tumors (e.g., hamartomas). These tumors can affect a variety of tissues, including the brain, heart, kidneys, skin, and lungs. In the brain, they can cause developmental delay, autism, epilepsy, and hydrocephalus. Life-threatening conditions in TSC include renal angiomyolipomas, which can cause internal bleeding, and lymphangioleiomyomatosis (LAM), which can compromise breathing. Although rapamycin and related drugs have been effective in reducing the size of lesions for some types of tumors, they must be administered continuously and have side effects, including compromised brain development and immune suppression. In addition, some patients do not respond to these medications, or respond initially and then become resistant. Accordingly, there exists a need in the art for improved treatments for TSC.

### SUMMARY OF THE INVENTION

The invention provides compositions and methods for treating tuberous sclerosis complex (TSC) caused by mutations in the TSC2 gene. The compositions and methods described herein relate to a condensed tuberin (cTuberin) and nucleic acid molecules encoding cTuberin.

In a first aspect, the invention features a cTuberin including a hamartin binding region and a GTPase-activating protein (GAP) region, but lacking an Akt phosphorylation site Thr 1462.

In some embodiments, the cTuberin has at least 90% sequence identity (e.g., 90%, 91%, 92%, 93%, 94%, 95%, 96%, 97%, 98%, or 99% sequence identity) to SEQ ID NO: 1. In particular embodiments, the cTuberin is SEQ ID NO: 1.

In some embodiments, the hamartin binding region has at least 90% sequence identity (e.g., 90%, 91%, 92%, 93%, 94%, 95%, 96%, 97%, 98%, or 99% sequence identity) to SEQ ID NO: 2. In particular embodiments, the hamartin binding region is SEQ ID NO: 2.

In some embodiments, the GAP region has at least 90% sequence identity (e.g., 90%, 91%, 92%, 93%, 94%, 95%, 96%, 97%, 98%, or 99% sequence identity) to SEQ ID NO: 3. In particular embodiments, the GAP region is SEQ ID NO: 3.

An engineered cTuberin further lacks an Akt phosphorylation site of human tuberin (SEQ ID NO: 10). In particular embodiments, cTuberin lacks amino acids 451-1514 of human tuberin (SEQ ID NO: 10) which includes an Akt phosphorylation site at Thr 1462 of human tuberin.

In yet other embodiments, the cTuberin includes a spacer between the hamartin binding region and GAP region. In some embodiments, the spacer includes at least SGGG. An exemplary spacer is SGGGSGGGSGGGSGGG (SEQ ID NO: 4).

In yet other embodiments, cTuberin is produced using a human tuberin isoform as is disclosed herein.

In a second aspect, the invention features a nucleic acid molecule encoding the cTuberin of any of the foregoing embodiments.

In other embodiments, the nucleic acid molecule is codon optimized for expression in a human cell (e.g., a brain cell, a heart cell, a kidney cell, a skin cell, or a lung cell).

In some embodiments, the nucleic acid molecule is operably linked to a regulatory control sequence. Exemplary regulatory control sequences include, without limitation, a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, or a glial fibrillary acidic protein (GFAP) promoter. In one working example, the regulatory control sequence includes CMV immediate/early gene enhancer/CBA promoter and a woodchuck hepatitis virus posttranscriptional regulatory element (WPRE).

In other embodiments, the nucleic acid molecule has at least 90% sequence identity (e.g., 90%, 91%, 92%, 93%, 94%, 95%, 96%, 97%, 98%, or 99% sequence identity) to SEQ ID NO: 5. In particular embodiments, the nucleic acid molecule is SEQ ID NO: 5.

In some embodiments, the nucleic acid molecule is operably linked to an expression cassette.

In a third aspect, the invention features a cell or virus including the nucleic acid molecule of any of the foregoing embodiments.

In a fourth aspect, the invention features a composition including the nucleic acid molecule of any of the foregoing embodiments.

In a fifth aspect, the invention features a recombinant adeno-associated virus (rAAV). Such a rAAV includes an AAV capsid and an AAV genome packaged therein, the AAV genome including a nucleic acid molecule capable of expressing cTuberin. For example, the rAAV includes an AAV capsid and an AAV genome packaged therein, the AAV genome including: (a) an AAV 5′ inverted terminal repeat (ITR) sequence; (b) a regulatory control sequence; (c) a nucleic acid molecule encoding cTuberin; and (d) an AAV 3′ ITR sequence. In another example, the cTuberin includes a hamartin binding region and a GAP region, but lacks an Akt phosphorylation site Thr 1462.

In some embodiments, the AAV capsid is an AAV1, AAV2, AAV3, AAV4, AAV5, AAV6, AAV7, AAV8, AAV9, AAV10, AAV11, or AAV12 capsid, or a variant of any one of the AAV capsids.

In other embodiments, the nucleic acid molecule is codon optimized for expression in a human cell. In further embodiments, the nucleic acid molecule has at least 90% sequence identity to SEQ ID NO: 5. In particular embodiments, the nucleic acid molecule is SEQ ID NO: 5.

In other embodiments, the nucleic acid is operably linked to a regulatory control sequence. Exemplary regulatory control sequences include, without limitation, a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, or a glial fibrillary acidic protein (GFAP) promoter.

In some embodiments, the nucleic acid molecule includes an inverted terminal repeat (ITR). In some embodiments, the nucleic acid molecule includes a polyadenylation signal such as a poly A region.

In a sixth aspect, the invention features a composition including the rAAV of any one of the foregoing embodiments and a pharmaceutically acceptable carrier.

In a seventh aspect, the invention features a method of treating a patient having tuberous sclerosis complex (TSC), the method including administering to the patient a cTuberin including a hamartin binding region and a GAP region, but lacking an Akt phosphorylation site Thr 1462.

In some embodiments, the patient is administered a nucleic acid molecule encoding cTuberin.

In some embodiments, the patient is administered a rAAV of any of the preceding aspects.

In some embodiments, the patient is administered extracellular vesicles (EVs) including the nucleic acid molecule of any of the preceding aspects.

In some embodiments, the patient has a renal angiomyolipoma. In some embodiments, the cTuberin is administered intravascularly or is administered into the renal artery or vein.

In other embodiments, the patient has a lymphangioleiomyomatosis (LAM). In some embodiments, the cTuberin is administered intravascularly or is administered into the lungs.

In yet other embodiments, the patient has a brain dysfunction. In some embodiments, the cTuberin is administered intravascularly, intracerebrally, or intrathecally.

In some embodiments, the cTuberin is administered to a renal angiomyolipoma, a LAM, or the brain.

In some embodiments, the rAAV is administered to a brain cell, a heart cell, a kidney cell, a skin cell, or a lung cell. In yet other embodiments, the rAAV is administered intravascularly, intravenously, intracerebrally, intraventricularly, intrathecally, or dermally.

In some embodiments, the patient is further administered a drug used to treat TSC. Such a drug may be rapamycin or a rapamycin analog.

### Definitions

As used herein, “administering” or a grammatical derivation thereof refers to the placement of an agent as disclosed herein into a subject by a method or route which results in at least partial localization of the agent at a desired site.

As used herein, “codon optimization” refers to modifying a nucleic acid sequence to change individual nucleic acids without any resulting change in the encoded amino acid. Sequences modified in this way are referred to herein as “codon optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15(20): 8125-8148 (1987), incorporated herein by reference in its entirety.

As used herein, a sequence which “encodes” a particular protein is a nucleic acid molecule that is transcribed (in the case of DNA) and translated (in the case of mRNA) into a polypeptide in vitro or in vivo when placed under the control of appropriate regulatory sequences; although one of skill in the art will readily appreciate that various polynucleotides do not operate in this fashion (e.g., antisense RNA, siRNA, ribozymes, wherein the RNA transcript is the product). With respect to protein products (i.e., not RNA products), the boundaries of the coding sequence are determined by a start codon at the 5′ (i.e., amino) terminus and a translation stop codon at the 3′ (i.e., carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence. Moreover, a “gene” (i) starts with a promoter region containing multiple regulatory elements, possibly including enhancers, for directing transcription of the coding region sequences; (ii) includes coding sequences, which start at the transcriptional start site that is located upstream of the translational start site and ends at the transcriptional stop site, which may be quite a bit downstream of the stop codon (a polyadenylation signal is usually associated with the transcriptional stop site and is located upstream of the transcriptional stop); and (iii) may contain introns and other regulatory sequences to modulate expression and improve stability of the RNA transcript. Still in accordance with the present invention, a “gene” may refers to a sequence encoding a protein.

As used herein, “expression” refers to the process by which a structural gene produces a polypeptide. It involves transcription of the gene into mRNA, and the translation of such mRNA into polypeptides(s).

As used herein, an “expression vector” is a vector or vehicle similar to a cloning vector but which is capable of expressing a gene which has been cloned into it after transformation into a host. The cloned gene is usually placed under the control of (i.e., operably linked to) certain control sequences such as promoter sequences. Expression control sequences will vary depending on whether the vector is designed to express the operably linked gene in a prokaryotic or eukaryotic host and can additionally contain transcriptional elements such as enhancer elements, termination sequences, tissue-specificity elements, and/or translational initiation and termination sites.

As used herein, “nucleic acid” or “nucleic acid molecule,” as generally understood and used herein, refers to chains of nucleotides joined together by phosphodiester bonds to form a nucleic acid heteropolymer. The nucleic acid molecules can be double stranded or single stranded and can be deoxyribonucleotide (DNA) molecules, such as cDNA or genomic DNA, or ribonucleotide (RNA) molecules. As such, the nucleic acid molecule can include one or more exons, with or without, as appropriate, introns.

As used herein, “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, “protein” and “polypeptide” are used interchangeably herein and refer to a polymer of amino acids. A peptide is a relatively short polypeptide, typically between about 2 and 60 amino acids in length. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

As used herein, “regulatory control element” or “regulatory control sequence” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. These control elements need not always be present, so long as the selected coding sequence is capable of being replicated, transcribed and translated in an appropriate host cell.

As used herein, “recombinant virus” refers to a virus that has been genetically altered (e.g., by the addition or insertion of a heterologous nucleic acid construct into the particle).

As used herein, the terms “subject” and “patient” are interchangeable and refer to an organism that receive treatment for a particular disease or condition as described herein.

As used herein, “treat,” “treatment,” “treating,” or “amelioration” are used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease-state is reduced or halted. That is, “treatment” includes not just the improvement of symptoms or markers, but also a cessation or at least slowing of progress or worsening of symptoms that would be expected in the absence of treatment. For example, in the case of renal angiomyolipomas, tumor size can be monitored by MRI and the shrinkage in cell size due to replacement of tuberin function can be revealed according to standard procedures (e.g., such as those used to monitor treatment of TSC using rapamycin).

Beneficial or desired clinical results include, but are not limited to, alleviation of one or more symptom(s), diminishment of extent of the deficit, stabilized (i.e., not worsening) state of tuberous sclerosis complex progression, delay or slowing of invasiveness or growth of tumors or hamartomas, and amelioration or palliation of symptoms associated with such tumors or hamartomas. Treatment also includes a decrease in mortality or an increase in the lifespan of a subject as compared to one not receiving the treatment.

As used herein, “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

Unless otherwise defined herein, scientific and technical terms used regarding the present application shall have the meanings that are commonly understood by those of ordinary skill in the art to which this disclosure belongs. This invention is not limited to the particular methodology, protocols, and reagents, etc., described herein and as such can vary. The terminology used herein is for describing particular embodiments only, and is not intended to limit the scope of the present invention, which is defined solely by the claims.

The invention described herein provides numerous advantages. For example, described herein are compositions and methods useful for treating tuberous sclerosis complex by gene therapy using recombinant adeno-associated viruses. Previously, TSC caused by mutations in TSC2 were not corrected with gene therapy due to the relatively small insert capacity of an AAV vector of 4.7 kb compared to the 5.4 kb cDNA of human tuberin. As is described herein, the disclosed methods for correcting mutations in TSC2 utilizes a condensed form of human tuberin, cTuberin. The cDNA of the cTuberin described herein is about 2.3 kb, which is readily expressed in an AAV vector. Indeed, AAV vectors have proven safe and beneficial in gene therapy. These vectors can be delivered, e.g., intravascularly to reach many tissues in a single injection, with some serotypes able to cross the blood brain barrier. Typically, a single injection confers beneficial outcome over a long term. Our compositions and methods allow for the use of AAV vectors expressing cTuberin for treatment of manifestations of tuberous sclerosis complex in patients with mutations in TSC2. Such vectors are not only useful for treating renal angiomyolipomas, but also for LAM and brain dysfunctions resulting from TSC2.

Further, the invention provides an alternative method of treating TSC not necessarily requiring rapamycin or its analogues, which may cause toxicity and adverse events related to over-suppression of mTORC1. Instead, the disclosed compositions and methods employ the functionality of tuberin, which is only active when complexed with hamartin. Because hamartin levels are normal in TSC2 patients, it is envisioned that there is little-to-low toxicity due to overexpression of cTuberin through vector delivery. Additionally, while rapamycin and related drugs can inhibit mTORC1 activity, cTuberin is capable of inhibiting both mTORC1 and mTORC1-independent Rheb-dependent pathological actions, and is therefore potentially more efficacious than previous therapies for tuberous sclerosis.

Other features and advantages of the invention will be apparent from the following description of the preferred embodiments thereof, and from the claims.

### BRIEF DESCRIPTION OF THE DRAWINGS

FIG. 1A shows the amino acid sequence of cTuberin (SEQ ID NO: 1).

FIG. 1B shows the nucleic acid sequence encoding cTuberin (SEQ ID NO: 5).

FIG. 2A is a schematic diagram of the functional domains of the TSC1 and TSC2 full-length human proteins and the condensed tuberin (cTuberin) protein. Amino acid residues are indicated by the numbers above the arrows. T2BD refers to the TSC2-binding domain, T1BD refers to the TSC1-binding domain, coil refers to the predicted coiled-coil domain, and GAP refers to GTPase-activating protein, which is a domain in human tuberin homologous to that in Rap1GAP.

FIG. 2B is a schematic diagram of the cTuberin AAV vector.

FIG. 2C is a schematic diagram of the AAV-CBA-cTuberin vector plasmid construct.

FIG. 3A is a Western blot showing the expression level of cTuberin in COS-7 cells transfected with the AAV-CBA-cTuberin vector plasmid construct. A band appears at the predicted molecular weight for cTuberin of approximately 85 kDa.

FIG. 3B is a Western blot showing the expression levels of pS6, S6, and GAPDH in cells transfected with various constructs. pS6 expression was elevated in the cells lacking cTuberin activity.

FIG. 4A is a graph showing the survival of Tsc2^{c/c} mice injected with AAV1-CBA-Cre vector at birth (postnatal day 0 (P0)), Tsc2^{c/c} mice injected with AAV9-CBA-cTuberin at P21, and non-injected mice. The median lifespan of the AAV1-CBA-Cre injected mice was 35 days, while the median lifespan was >185 days for the AAV9-CBA-cTuberin injected mice and >175 days for the non-injected mice.

FIG. 4B is a graph showing the survival of Tsc2^{c/c} mice injected with AAV1-CBA-Cre vector at P0 and mice injected with AAV1-CBA-Cre vector at P0 followed by injection with AAV1-CBA-cTuberin vector at P21. The median lifespan was 26.5 days for the non-injected mice and 35 days for the AAV1-CBA-cTuberin injected mice.

FIG. 4C is a graph showing the survival of Tsc2^{c/c} mice injected with AAV1-CBA-Cre vector at P3 and mice injected with AAV1-CBA-Cre vector at P3 followed by injection with AAV1-CBA-cTuberin vector at P21. The median lifespan was 36 days for the non-injected mice and 54 days for the AAV1-CBA-cTuberin injected mice.

FIG. 4D is a graph showing the survival of Tsc2^{c/c} mice injected with AAV1-CBA-Cre vector at P3 and mice injected with AAV1-CBA-Cre vector at P3 followed by injection with AAV9-CBA-cTuberin vector at P21. The median lifespan was 32 days for the non-injected mice and 45 days for the AAV9-CBA-cTuberin injected mice.

FIG. 4E is a graph showing the survival of four groups of Tsc2^{c/c} mice. The first group was injected with AAV1-CBA-Cre vector at P0 only; the second group was injected with AAV1-CBA-Cre vector at P0 followed by injection with AAV9-CBA-cTuberin at P3; the third group mice was injected with AAV1-CBA-Cre vector at P0 only and treated with vigabatrin; and the fourth group was injected with AAV1-CBA-Cre vector at P0 followed by injection with AAV9-CBA-cTuberin at P3 and treated with vigabatrin. The median lifespan of non-injected, no drug mice was 23 days; of non-injected, vigabatrin-treated mice was 27 days; of injected, no drug mice was 35.5 days; and injected, vigabatrin-treated mice was 40 days.

FIGS. 5A-5F shows the staining of mice brains treated according to the experimental design of FIG. 4B using Hematoxylin and Eosin (H&E) staining or immunohistochemistry (IHC) for pS6. FIG. 5A show the staining in a normal, non-injected mice (control); FIGS. 5B-5E show the staining in mice injected at P0 with AAV1-CBA-Cre vector, and FIG. 5F shows the staining for the mice further treated at P3 with AAV1-CBA-cTuberin.

FIG. 6A shows the volumes of the lymphangioleiomyomatosis (LAM) tumors injected subcutaneously into NOD-SCID II2R gamma (NSG) mice.

FIG. 6B is a graph showing the growth of LAM tumors in non-injected mice and mice injected with AAV9-CBA-cTuberin at weeks 4 and 9.

### DETAILED DESCRIPTION OF THE EMBODIMENTS OF THE INVENTION

Systemic gene therapy, as is disclosed herein, can be achieved in TSC patients by delivery (e.g., via the vascular system) of a condensed tuberin (cTuberin) such as one encoded in an AAV vector, which is useful for decreasing the size of affected cells and reduction of hamartomas in multiple tissues, including throughout the central nervous system, kidneys, and lungs. cTuberin is useful, for example, for its ability to suppress mTOR activity. Useful forms of cTuberin may be identified according to any method, e.g., by testing their ability to suppress S6 phosphorylation in cells in vitro lacking tuberin as is described herein. Below we describe a useful design of a cTuberin in treating TSC.

In the Examples below, we describe engineering a condensed version of human tuberin, termed cTuberin, which fits into an AAV vector. The size of human tuberin cDNA is 5.4 kb, exceeding the transgene packaging capacity of AAV. To this end, engineering our condensed form of tuberin was accomplished by deleting the central portion of the human tuberin cDNA. Our cTuberin retains the hamartin binding region in the N-terminal and the GAP region in the C-terminal of human tuberin, but lacks an Akt phosphorylation site Thr1462, such that Akt activation of mTORC1 is decreased or eliminated. The central region of the protein is replaced by a glycine-serine linker to confer conformational flexibility. This cTuberin cDNA was then cloned into an AAV vector under a strong ubiquitous chicken beta actin promoter. We have transduced mouse embryonic fibroblasts with this AAV-CBA-cTuberin construct and as is shown by Western blot analysis that it reduces S6 kinase activity, which is a marker of mTORC1 activation, thus establishing biologic activity of cTuberin.

I. cTuberin

cTuberin, as is described herein, in general, includes a hamartin binding region, a GTPase-activating protein (GAP) region, and a spacer linking the hamartin binding region to the GAP region, and lacks the Akt phosphorylation site at Thr 1462 of human tuberin.

An exemplary cTuberin useful for treating TSC has the amino acid sequence of SEQ ID NO: 1. In this cTuberin (SEQ ID NO: 1), the amino acid sequence of the hamartin binding region is SEQ ID NO: 2. Also in this cTuberin (SEQ ID NO: 1), the amino acid sequence of the GAP region is SEQ ID NO: 3.

The hamartin binding region and GAP region of this cTuberin (SEQ ID NO: 1) are connected by a protein spacer sequence. In one example, the spacer sequence includes a glycine-serine (SGGG) linker sequence, e.g., SEQ ID NO: 4. In this instance, a 16 a.a. linker connects the hamartin binding region and the GAP region. The cTuberin protein relative to human tuberin (SEQ ID NO: 10) lacks the Akt phosphorylation site Thr 1462 of human tuberin, which is one of several phosphorylation sites involved in the regulation of tuberin activity (Huang et al., Biochem. J. 412(2):179-190 2008).

In another example, cTuberin has at least 80% sequence identity (e.g., 80%, 85%, 90%, 91%, 92%, 93%, 94%, 95%, 96%, 97%, 98%, or 99% sequence identity) to SEQ ID NO: 1. Of this cTuberin, the hamartin binding region of cTuberin has at least 80% sequence identity (e.g., 80%, 85%, 90%, 91%, 92%, 93%, 94%, 95%, 96%, 97%, 98%, or 99% sequence identity) to SEQ ID NO: 2, and the GAP region of cTuberin has at least 80% sequence identity (e.g., 80%, 85%, 90%, 91%, 92%, 93%, 94%, 95%, 96%, 97%, 98%, or 99% sequence identity) to SEQ ID NO: 3. In some cTuberins, the hamartin binding region and GAP region of cTuberin are connected by a protein spacer sequence. In one example, the spacer sequence includes a glycine-serine (SGGG) linker sequence, e.g., SEQ ID NO: 4.

The cTuberin of SEQ ID NO: 1 described herein was produced using human tuberin, the amino acid and nucleic acid sequences of which can be found at NCBI Accession No. NP_000539.2 and GenBank Accession No. X75621.1, respectively. Other human tuberin isoforms may also be used for producing additional cTuberins. Exemplary human tuberin isoforms useful to produce such molecules include, but are not limited to, tuberin isoform 4 (NCBI Accession No. NP_001070651.1), tuberin isoform 5 (NCBI Accession No. NP_001107854.1), tuberin isoform 6 (NCBI Accession No. NP_001305756.1), tuberin isoform 7 (NCBI Accession No. NP_001305758.1), tuberin isoform 8 (NCBI Accession No. NP_001305760.1), tuberin isoform 9 (NCBI Accession No. NP_001305761.1), tuberin isoform X7 (NCBI Accession No. XP_024306181.1), tuberin isoform X8 (NCBI Accession No. XP_005255586.2), tuberin isoform X9 (NCBI Accession No. XP_016879105.1), tuberin isoform X10 (NCBI Accession No. XP_005255588.2), tuberin isoform X11 (NCBI Accession No. XP_016879106.1), tuberin isoform X12 (NCBI Accession No. XP_016879107.1), and others. Such tuberins are useful for engineering any cTuberin as described herein.

For example, a cTuberin can be engineered using human tuberin isoform 4 (NCBI Accession No. NP_001070651.1). Using human tuberin isoform 4, the amino acid sequence of a hamartin binding region having at least 80% (e.g., 80%, 85%, 90%, 91%, 92%, 93%, 94%, 95%, 96%, 97%, 98%, or 99% sequence identity) sequence identity to SEQ ID NO: 2 and the amino acid sequence of a GAP region having at least 80% (e.g., 80%, 85%, 90%, 91%, 92%, 93%, 94%, 95%, 96%, 97%, 98%, or 99% sequence identity) sequence identity to SEQ ID NO: 3 are produced as described for cTuberin (SEQ ID NO:1). Further, the hamartin binding and GAP regions can be connected by a protein spacer sequence. In one example, the spacer sequence includes a glycine-serine (SGGG) linker sequence, e.g., SEQ ID NO: 4. A cTuberin engineered from human tuberin isoform 4 further lacks an Akt phosphorylation site.

II. cTuberin Nucleic Acid Molecules

Further, the exemplary cTuberin of SEQ ID NO: 1 is encoded by a nucleic acid molecule having the sequence of SEQ ID NO: 5. In this cTuberin nucleic acid molecule (SEQ ID NO: 5), the hamartin binding region is encoded by SEQ ID NO: 6. Also in this cTuberin nucleic acid molecule (SEQ ID NO: 5), the GAP region is encoded by SEQ ID NO: 7.

In this exemplary cTuberin of SEQ ID NO: 1, encoded by the nucleic acid molecule of SEQ ID NO: 5, the hamartin binding region and GAP region are linked by a protein spacer sequence, i.e., a glycine-serine linker of SEQ ID NO: 4. The glycine-serine linker is encoded by SEQ ID NO: 8.

In a further embodiments, the cTuberin encoding nucleic acid has at least 80% sequence identity (e.g., 80%, 85%, 90%, 91%, 92%, 93%, 94%, 95%, 96%, 97%, 98%, or 99% sequence identity) to SEQ ID NO: 5. Of this cTuberin, the hamartin binding region is encoded by a nucleic acid having at least at least 80% sequence identity (e.g., 80%, 85%, 90%, 91%, 92%, 93%, 94%, 95%, 96%, 97%, 98%, or 99% sequence identity) to SEQ ID NO: 6, and the GAP region is encoded by a nucleic acid having at least at least 80% sequence identity (e.g., 80%, 85%, 90%, 91%, 92%, 93%, 94%, 95%, 96%, 97%, 98%, or 99% sequence identity) to SEQ ID NO: 7. Further, the cTuberin encoding nucleic acid may include a protein spacer encoding sequence, for example, SEQ ID NO: 8.

The cTuberin nucleic acid molecule may be codon optimized for expression in a human cell. Further, the cTuberin nucleic acid molecule may be operably linked to a regulatory control sequence, such as, for example, a CMV immediate/early gene enhancer/CBA promoter and a woodchuck hepatitis virus posttranscriptional regulatory element (WRPE), or, without limitation, a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, or a glial fibrillary acidic protein (GFAP) promoter. The nucleic acid molecule may also be operably linked to an expression cassette.

For example, the cTuberin nucleic acid molecule (SEQ ID NO: 5) may be inserted under a CBA promoter with a Kozak sequence followed by a wood chuck hepatitis virus post-transcriptional regulatory element (WPRE) and a SV40 and bovine growth hormone polyadenylation signal sequence. The vector is inserted into an AAV2-LTR backbone and is flanked by AAV2 ITR sequences.

Further, the cTuberin nucleic acid molecule (SEQ ID NO: 5) may be incorporated into a vector plasmid genome. An exemplary vector plasmid genome that includes the cTuberin nucleic acid molecule (SEQ ID NO: 5) has the sequence of SEQ ID NO: 11.

### III. Recombinant AAV Molecules

Any suitable nucleic acid vector may be used in conjunction with the present compositions and methods to design and assemble the components of a nucleic acid molecule encoding cTuberin and a recombinant adeno-associated virus (AAV). rAAV vectors useful in the compositions and methods described herein are recombinant nucleic acid constructs that include (1) a heterologous sequence to be expressed (e.g., a nucleic acid molecule encoding cTuberin) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part, but retain functional flanking ITR sequences. The AAV ITRs may be of any serotype suitable for a particular application. Methods for using rAAV vectors are described, for example, in Tal et al., J. Biomed. Sci. 7:279-291 (2000), and Monahan et al., Gene Therapy. 7:24-30 (2000), the disclosures of each of which are incorporated herein by reference as they pertain to AAV vectors for gene delivery.

In one embodiment, the vector is a recombinant AAV carrying the cTuberin nucleic acid molecule and driven by a promoter that expresses a cTuberin molecule in selected cells of a subject. Methods for assembly of the recombinant vectors are known in the art. See, e.g., Ausubel et al., Current Protocols in Molecular Biology, John Wiley & Sons, New York, 1989; Kay, M. A. et al., Nat. Med. 7(1):33-40 (2001); and Walther W. and Stein U., Drugs 2000, 60(2):249-71.

In certain embodiments described herein, the cTuberin nucleic acid molecule is delivered to the selected cells, e.g., a brain, heart, kidney, skin, or lung cell, in need of treatment by means of an AAV vector according to standard methods known in the art. More than 30 naturally occurring serotypes of AAV are available. Many natural variants in the AAV capsid exist, allowing identification and use of an AAV with properties specifically suited for the selected cells. AAV viruses may be engineered by conventional molecular biology techniques, making it possible to optimize these particles for cell specific delivery of the cTuberin nucleic acid molecule sequences, for minimizing immunogenicity, for tuning stability and particle lifetime, for efficient degradation, for accurate intracellular delivery, e.g., to the nucleus.

The expression of the cTuberin nucleic acid molecules described herein can be achieved in the selected cells through delivery by recombinantly engineered AAVs or artificial AAVs that contain sequences encoding the desired cTuberin nucleic acid molecule. The use of AAVs is a common mode of exogenous delivery of DNA as it is relatively non-toxic, provides efficient gene transfer, and can be easily optimized for specific purposes. Among the well-characterized serotypes of AAVs isolated from human or non-human primates, human serotype 2 has been widely used for efficient gene transfer experiments in different target tissues and animal models. Other AAV serotypes include, but are not limited to, AAV1, AAV3, AAV4, AAV5, AAV6, AAV7, AAV8, AAV9, AAV10, AAV11, AAV12, or a hybrid serotype thereof. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV11, AAV12, a hybrid serotype thereof, or other known and unknown AAV serotypes. In one embodiment, the ITRs are from AAV2. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

Desirable AAV fragments for assembly into vectors include the cap proteins, including the vp1, vp2, vp3, and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid. Pseudotyped vectors, wherein the capsid of one AAV is utilized with the ITRs from an AAV having a different capsid protein, are useful in the compositions and methods described herein.

In one example, the AAV includes a capsid sequence derived from AAV1. In another embodiment, the AAV includes a capsid sequence derived from AAV9. The use of AAV1 and AAV9 have been previously described in Broekman et al., Neuroscience. 138:501-510, 2006, which is incorporated herein by reference.

In another example, the vectors useful in compositions and methods described herein contain, sequences encoding a selected AAV serotype capsid, e.g., an AAV1 or AAV9 capsid, or a fragment thereof. Other useful vectors contain, sequences encoding a selected AAV serotype rep protein, e.g., AAV1 or AAV9 rep protein, or a fragment thereof. Optionally, such vectors may contain both AAV cap and rep proteins. In vectors in which both AAV rep and cap are provided, the AAV rep and AAV cap sequences can both be of one serotype origin, e.g., an AAV1 or AAV9 origin.

Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector described in U.S. Pat. No. 7,282,199, which is incorporated by reference herein.

A suitable recombinant AAV (rAAV) is generated by culturing a host cell which contains a nucleic acid sequence encoding an AAV serotype capsid protein, or fragment thereof, as defined herein; a functional rep gene; a minigene composed of, e.g., AAV ITRs and the cTuberin nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In one working example, the AAV includes a promoter (or a functional fragment of a promoter). The selection of the promoter to be employed in the rAAV may be made from among a wide number of constitutive or inducible promoters that can express the selected transgene in the desired target cell, which are known in the art. In one embodiment, the promoter is cell-specific. The term “cell-specific” means that the particular promoter selected for the recombinant vector can direct expression of the selected transgene in a particular cell type. In one embodiment, the promoter is specific for expression of the transgene in a brain cell, a heart cell, a kidney cell, a skin cell, or a lung cell.

In another embodiment, the promoter is the native promoter for the target gene to be expressed. Useful promoters include, without limitation, human cytomegalovirus (CMV) promoter, chicken β-actin (CBA) promoter, Rous sarcoma virus (RSV) LTR promoter/enhancer, SV40 promoter, dihydrofolate reductase promoter, phosphoglycerol kinase promoter, CMV-immediate early (IE) enhancer/CBA promoter, synapsin promoter, and glial fibrillary acidic protein (GFAP) promoter.

Other conventional regulatory sequences contained in the minigene or rAAV are known in the art. One of skill in the art may make a selection among these, and other, expression control sequences without departing from the scope described herein

An AAV minigene may include the cTuberin nucleic acid molecule described herein and its regulatory sequences, and 5′ and 3′ AAV ITRs. In one embodiment, the ITRs of AAV serotype 2 are used. However, ITRs from other suitable serotypes may be selected. In some embodiments, the minigene is packaged into a capsid protein and delivered to a selected host cell.

The minigene, rep sequences, cap sequences, and helper functions required for producing the rAAV may be delivered to the packaging host cell in the form of any genetic element which transfers the sequences carried thereon. The selected genetic element may be delivered by any suitable method, including those described herein. The methods used to construct any embodiment described herein are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al., J. Virol., 1993 70: 520-532 and U.S. Pat. No. 5,478,745, each of which is incorporated by reference herein.

In another working example, a cTuberin minigene is prepared in a proviral plasmid, such as those disclosed in International Patent Publication No. WO 2012/158757, incorporated herein by reference. Such a proviral plasmid contains a modular recombinant AAV genome comprising in operative association comprising: a wildtype 5′ AAV2 ITR sequence flanked by unique restriction sites that permit ready removal or replacement of said ITR; a promoter comprising a cytomegalovirus sequence upstream of a cytomegalovirus (CMV)-chicken beta actin sequence, or a cell-specific promoter/enhancer, the promoter flanked by unique restriction sites that permit ready removal or replacement of the entire promoter sequence, and the upstream sequence flanked by unique restriction sites that permit ready removal or replacement of only the upstream CMV or enhancer sequence, from the promoter sequence. The cTuberin nucleic acid molecule described herein can be inserted into the site of a multi-cloning poly linker, wherein the cTuberin nucleic acid molecule is operatively linked to, and under the regulatory control of, the promoter. A bovine growth hormone polyadenylation sequence flanked by unique restriction sites that permit ready removal or replacement of said poly A sequence; and a wildtype 3′ AAV2 ITR sequence flanked by unique restriction sites that permit ready removal or replacement of the 3′ ITR; are also part of this plasmid. The plasmid backbone comprises the elements necessary for replication in bacterial cells and is itself flanked by transcriptional terminator/insulator sequences.

In yet another working example, a proviral plasmid comprises a modular recombinant AAV genome comprising in operative association comprising: (i) a wildtype 5′ AAV2 ITR sequence flanked by unique restriction sites that permit ready removal or replacement of said ITR; (ii) a promoter comprising (A) a CMV immediate/early enhancer sequence upstream of a CMV-chicken beta actin sequence; or (B) a cell-specific promoter/enhancer including, for example, a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, or a glial fibrillary acidic protein (GFAP) promoter, and others. The promoter is flanked by unique restriction sites that permit ready removal or replacement of the entire promoter sequence, and the upstream sequence flanked by unique restriction sites that permit ready removal or replacement of only the upstream CMV or enhancer sequence, from the promoter sequence. Also part of this proviral plasmid is a multi-cloning polylinker sequence that permits insertion of a cTuberin nucleic acid sequence including any of those described herein, wherein the cTuberin nucleic acid molecule is operatively linked to, and under the regulatory control of, the promoter; a bovine growth hormone polyadenylation sequence flanked by unique restriction sites that permit ready removal or replacement of said poly A sequence; and a wildtype 3′ AAV2 ITR sequence flanked by unique restriction sites that permit ready removal or replacement of the 3′ ITR. The proviral plasmid also contains a plasmid backbone comprising the elements necessary for replication in bacterial cells, and further comprising a kanamycin resistance gene, said plasmid backbone flanked by transcriptional terminator/insulator sequences. The proviral plasmid described herein may also contain in the plasmid backbone a non-coding lambda phage 5.1 kb stuffer sequence to increase backbone length and prevent reverse packaging of non-functional AAV genomes.

In some embodiments, a proviral plasmid contains multiple copies of a cTuberin nucleic acid molecule. For example, cTuberin nucleic acid molecules that are less than half the packaging limit for AAV can therefore be repeated once, twice, three times, four times, five times, six times, seven times, eight times, nine times, 10 times, 11 times, 12 times, 13 times, 14 times, 15 times, 16 times, 17 times, 18 times, 19 times, 20 times, or more on a single proviral plasmid.

In yet a further aspect, the promoter of the proviral plasmid is modified to reduce the size of the promoter to permit larger cTuberin nucleic acid molecule sequences to be inserted in the rAAV. In one embodiment, the CMV/CBA hybrid promoter, which normally includes a non-coding exon and intron totaling about 1,000 base pairs, is replaced with a 130-base pair chimeric intron, as is known in the art.

These proviral plasmids are then employed in currently conventional packaging methodologies to generate a recombinant virus expressing the cTuberin molecule transgene carried by the proviral plasmids. Suitable production cell lines are readily selected by one of skill in the art. For example, a suitable host cell can be selected from any biological organism, including prokaryotic (e.g., bacterial) cells, and eukaryotic cells, including, insect cells, yeast cells and mammalian cells. Briefly, the proviral plasmid is transfected into a selected packaging cell, where it may exist transiently. Alternatively, the minigene or gene expression cassette with its flanking ITRs is stably integrated into the genome of the host cell, either chromosomally or as an episome. Suitable transfection techniques are known and may readily be utilized to deliver the recombinant AAV genome to the host cell. Typically, the proviral plasmids are cultured in the host cells which express the cap and/or rep proteins. In the host cells, the minigene consisting of the cTuberin nucleic acid molecule with flanking AAV ITRs is rescued and packaged into the capsid protein or envelope protein to form an infectious viral particle. Thus, a recombinant AAV infectious particle is produced by culturing a packaging cell carrying the proviral plasmid in the presence of sufficient viral sequences to permit packaging of the gene expression cassette viral genome into an infectious AAV envelope or capsid.

### IV. Extracellular Vesicles

Extracellular vesicles (EVs) are useful in the methods and compositions described herein. For example, EVs including any cTuberin described herein can be administered to a subject according to standard methods. In a further example, EVs including any nucleic acid molecule encoding cTuberin can be administered to a subject as well.

Extracellular vesicles, including but not limited to exosomes, microvesicles, microparticles, circulating microvesicles, shedding microvesicles, nanovesicles, nanoparticles, apoptotic bodies, and membrane vesicles, are fragments of plasma membrane ranging from for example, 20 nm to 10 μm, shed from almost all cell types. Microvesicles play a role in intercellular communication and can transport mRNA, miRNA, and proteins between cells. As will be apparent to a person of skill in the art, there are various EV isolation and purification protocols based on filtration, differential centrifugation, ultracentrifugation, flotation of vesicles in gradients (sucrose, OptiPrep™), and immunoaffinity capture utilizing antibodies against membrane proteins. Exemplary information for isolating extracelluar vesicles may be found in Simpson R J, Mathivanan S (2012) Extracellular Microvesicles: The Need for Internationally Recognised Nomenclature and Stringent Purification Criteria. J Proteomics Bioinform 5: ii-ii; van der Pol et al., Classification, functions, and clinical relevance of extracellular vesicles, Pharmacol Rev. 2012 July; 64(3):676-705; Raposo and Stoorvogel, Extracellular vesicles: exosomes, microvesicles, and friends, J Cell Biol. 2013 Feb. 18; 200(4):373-83; and Witwer et al., Standardization of sample collection, isolation and analysis methods in extracellular vesicle research, J Extracell Vesicles. 2013 May 27; 2, which are incorporated herein by reference in their entirety. Also, see Sarkar el al., 2009, Taylor and Gercel-Taylor, 2008, and Balaj et al., 2011, which are incorporated herein by reference in their entirety.

Typically, EVs are loaded according to standard procedures with any of the cTuberins described herein. For example, the EV can be loaded with the cTuberin of SEQ ID NO: 1.

Similarly, EVs are loaded with any of the nucleic acid molecules encoding cTuberin described herein. The nucleic acid molecule may be incorporated into an AAV genome. Further, the nucleic acid molecule can be operably linked to a regulatory control sequence including, for example, a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, or a glial fibrillary acidic protein (GFAP) promoter. In one example, the regulatory control sequence includes CMV immediate/early gene enhancer/CBA promoter and a woodchuck hepatitis virus posttranscriptional regulatory element (WPRE). In a further example, the nucleic acid molecule can include an ITR. In another example, the nucleic acid molecule includes a poly A.

Any EVs as described herein may also be included in a composition with a pharmaceutically acceptable carrier.

### V. Pharmaceutical Compositions and Kits

Provided herein are pharmaceutical compositions including a cTuberin nucleic acid molecule, EVs that includes a cTuberin nucleic acid molecule described herein (e.g., a rAAV), or a rAAV including a cTuberin nucleic acid molecule as is described herein. Such pharmaceutical compositions include any of the cTuberin nucleic acid molecules or cTuberins described herein.

The pharmaceutical compositions described herein may be assessed for contamination by conventional methods and then formulated into a pharmaceutical composition intended for a suitable route of administration. Still other compositions containing a cTuberin nucleic acid molecule, EVs comprising a cTuberin nucleic acid molecule, or a rAAV comprising a cTuberin nucleic acid molecule, may be formulated similarly with a suitable carrier. Such formulation involves the use of a pharmaceutically and/or physiologically acceptable vehicle or carrier, particularly directed for administration to the target cell. In one embodiment, carriers suitable for administration to the target cells include buffered saline, an isotonic sodium chloride solution, or other buffers, e.g., HEPES, to maintain pH at appropriate physiological levels, and, optionally, other medicinal agents, pharmaceutical agents, stabilizing agents, buffers, carriers, adjuvants, diluents, etc.

Typically, the carrier is a liquid for injection. Exemplary physiologically acceptable carriers include sterile, pyrogen-free water and sterile, pyrogen-free, phosphate buffered saline. In one embodiment, the carrier is an isotonic sodium chloride solution. In other examples, the carrier is a balanced salt solution. Other carriers include Tween. If the virus is to be stored long-term, it may be frozen in the presence of glycerol or Tween20.

Compositions containing cTuberin nucleic acid molecules described herein may also include a surfactant. Useful surfactants, such as Pluronic F68 (Poloxamer 188, also known as LUTROL® F68) may be included as they prevent AAV from sticking to inert surfaces and thus ensure delivery of the desired dose. As an example, one illustrative composition designed for the treatment of the diseases or disorders caused by a mutation in TSC2, e.g., tuberous sclerosis complex, as described herein comprises a recombinant adeno-associated vector carrying a nucleic acid sequence encoding cTuberin as described herein, under the control of regulatory sequences which express the cTuberin nucleic acid molecule in a brain cell, a heart cell, a kidney cell, a skin cell, or a lung cell of a mammalian subject, and a pharmaceutically acceptable carrier. The carrier is isotonic sodium chloride solution and includes a surfactant Pluronic F68. In one embodiment, the cTuberin nucleic acid molecule is any of those described herein.

In yet another exemplary embodiment, the composition includes a recombinant AAV1 or AAV9 pseudotyped adeno-associated virus carrying a cTuberin nucleic acid molecule for replacement, the nucleic acid sequence under the control of promoter which directs expression of the cTuberin nucleic acid molecule in brain, heart, kidney, skin, or lung cells, wherein the composition is formulated with a carrier and additional components suitable for dermal administration or intravascular, intracerebroventricular, intracranial, or intrathecal injection. In still another embodiment, the composition or components for production or assembly of this composition, including carriers, rAAV particles, surfactants, and/or the components for generating the rAAV, as well as suitable laboratory hardware to prepare the composition, may be incorporated into a kit.

Additionally, provided herein are kits containing a first pharmaceutical composition including a cTuberin nucleic acid molecule and a second pharmaceutical composition including drugs used for the treatment of tuberous sclerosis complex including, for example, rapamycin and its analogues. In some embodiments, the kit includes instructions for mixing the two pharmaceutical compositions prior to administration.

### VI. Methods

The compositions described above are useful in methods of treating diseases or disorders caused by a mutation in TSC2 by replacing a normal allele of TSC2. Such methods involve contacting a target TSC2 gene with a cTuberin nucleic acid molecule as described herein, under conditions in which the cTuberin nucleic acid molecule is delivered to a selected cell to correct expression of TSC2 in the target cell. Thus, the methods and compositions are used to treat the diseases or disorders caused by a mutation in the TSC2 associated with the specific mutations and/or gene expression.

In some embodiments, a cTuberin nucleic acid molecule, EVs including a cTuberin nucleic acid molecule, or a rAAV including a cTuberin nucleic acid molecule is administered to a brain cell, a heart cell, a kidney cell, a skin cell, or a lung cell. In some embodiments, a cTuberin nucleic acid molecule, EVs including a cTuberin nucleic acid molecule, or a rAAV including a cTuberin nucleic acid molecule is administered to the affected subject dermally, or by intravascular, intracerebroventricular, intracranial, or intrathecal injection.

In some embodiments, the methods include the administration of a cTuberin nucleic acid molecule, EVs including a cTuberin nucleic acid molecule, or a rAAV including a cTuberin nucleic acid molecule for treating a subject having a disorder associated with a mutation in TSC2, such as tuberous sclerosis complex. Such selection can be based on the genotype of the subject. In some embodiments, a disorder associated with TSC2 may be an autosomal dominant disorder. In some instances, the subject is homozygous or compound heterozygous for the mutation in TSC2. Methods of screening for and identifying particular mutations in TSC2 are known in the art.

### Dosing and Combination Therapies

Standards methods of dosing are used herein. Further, dosing for treatment of a renal angiomyolipoma, a lymphangioleiomyomatosis (LAM), and a brain dysfunction, as well as administration to a brain cell, heart cell, kidney cell, skin cell, or lung cell, are described below. Also described are dosing methods for administration by dermal, intravascular, intracerebral, intraventricular, or intrathecal injection.

An effective concentration of a recombinant adeno-associated virus carrying a cTuberin nucleic acid molecule as described herein ranges between about 10⁹ and 10¹⁵ genome copies (gc) per kg of body weight of the subject (gc/kg). For example, the effective concentration ranges between 10⁹ and 10¹⁵ gc/kg, e.g., 10⁹, 10¹⁰, 10¹¹, 10¹², 10¹³, 10¹⁴, and 10¹⁵ gc/kg. In another example, the effective concentration ranges between 10¹⁰ and 10¹³ gc/kg, e.g., 10¹⁰, 10¹¹, 10¹², and 10¹³ gc/kg. Still other dosages in these ranges or in other units may be selected by the attending physician, taking into account the physical state of the subject being treated, including the age of the subject; the composition being administered, and the particular disorder; the targeted cell and the degree to which the disorder, if progressive, has developed.

### Renal Angiomyolipoma

For example, a rAAV carrying a cTuberin nucleic acid molecule can be used to treat a patient with a renal angiomyolipoma. The rAAV can be administered to the patient according to any method, e.g., by intravascular injection, for example, into the renal artery or vein. The effective dosage of the rAAV for treatment of renal angiomyolipoma by intravascular injection is between 10⁹ and 10¹⁵ gc/kg. In one embodiment, the amount of rAAV administered to the patient is about 10⁹ gc/kg. In a further embodiment, the amount of rAAV administered to the patient is about 10¹⁰ gc/kg. In a further embodiment, the amount of rAAV administered to the patient is about 10¹¹ gc/kg. In a further embodiment, the amount of rAAV administered to the patient is about 10¹² gc/kg. In a further embodiment, the amount of rAAV administered to the patient is about 10¹³ gc/kg. In a further embodiment, the amount of rAAV administered to the patient is about 10¹⁴ gc/kg. In a further embodiment, the amount of rAAV administered to the patient is about 10¹⁵ gc/kg.

### LAM

A rAAV carrying a cTuberin nucleic acid molecule can also be used to treat a patient with a lymphangioleiomyomatosis (LAM). The rAAV can be administered to the patient according to any method, e.g., by intravascular injection. The effective dosage of the rAAV for treatment of a LAM by intravascular injection is between 10⁹ and 10¹⁵ gc/kg. In one embodiment, the amount of rAAV administered to the patient is about 10⁹ gc/kg. In a further embodiment, the amount of rAAV administered to the patient is about 10¹⁰ gc/kg. In a further embodiment, the amount of rAAV administered to the patient is about 10¹¹ gc/kg. In a further embodiment, the amount of rAAV administered to the patient is about 10¹² gc/kg. In a further embodiment, the amount of rAAV administered to the patient is about 10¹³ gc/kg. In a further embodiment, the amount of rAAV administered to the patient is about 10¹⁴ gc/kg. In a further embodiment, the amount of rAAV administered to the patient is about 10¹⁵ gc/kg. Further, the rAAV can administered to a patient with a LAM via a nasal route or other medically approved route into the lungs.

### Brain Dysfunction

In another example, a rAAV carrying a cTuberin nucleic acid molecule can be used to a patient with a brain dysfunction. The rAAV can be administered to the patient according to any method, e.g., by intravascular, intracerebroventricular, intracranial, or intrathecal injection.

For treatment of a brain dysfunction by intravascular injection, the effective dosage of the rAAV is between 10⁹ and 10¹⁵ gc/kg. In one embodiment, the amount of rAAV administered to the patient is about 10⁹ gc/kg. In a further embodiment, the amount of rAAV administered to the patient is about 10¹⁰ gc/kg. In a further embodiment, the amount of rAAV administered to the patient is about 10¹¹ gc/kg. In a further embodiment, the amount of rAAV administered to the patient is about 10¹² gc/kg. In a further embodiment, the amount of rAAV administered to the patient is about 10¹³ gc/kg. In a further embodiment, the amount of rAAV administered to the patient is about 10¹⁴ gc/kg. In a further embodiment, the amount of rAAV administered to the patient is about 10¹⁵ gc/kg.

Further, for treatment of a brain dysfunction by intracerebroventricular injection, the effective dosage of the rAAV is between 10¹⁰ and 10¹³ gc/kg. In one embodiment, the amount of rAAV administered to the patient is about 10¹⁰ gc/kg. In a further embodiment, the amount of rAAV administered to the patient is about 10¹¹ gc/kg. In a further embodiment, the amount of rAAV administered to the patient is about 10¹² gc/kg. In a further embodiment, the amount of rAAV administered to the patient is about 10¹³ gc/kg.

Further, for treatment of a brain dysfunction by intracranial injection, the effective dosage of the rAAV is between 10¹⁰ and 10¹³ gc/kg. In one embodiment, the amount of rAAV administered to the patient is about 10¹⁰ gc/kg. In a further embodiment, the amount of rAAV administered to the patient is about 10¹¹ gc/kg. In a further embodiment, the amount of rAAV administered to the patient is about 10¹² gc/kg. In a further embodiment, the amount of rAAV administered to the patient is about 10¹³ gc/kg.

Further, for treatment of a brain dysfunction by intrathecal injection, the effective dosage of the rAAV is between 10¹⁰ and 10¹³ gc/kg. In one embodiment, the amount of rAAV administered to the patient is about 10¹⁰ gc/kg. In a further embodiment, the amount of rAAV administered to the patient is about 10¹¹ gc/kg. In a further embodiment, the amount of rAAV administered to the patient is about 10¹² gc/kg. In a further embodiment, the amount of rAAV administered to the patient is about 10¹³ gc/kg.

### Delivery

The composition may be delivered in a volume of from about 50 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 350 μL. In another embodiment, the volume is about 400 μL In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1,000 μL.

In one embodiment, the volume and concentration of the rAAV composition is selected so that only certain anatomical regions having target cells are impacted. In another embodiment, the volume and/or concentration of the rAAV composition is a greater amount, in order reach larger portions of the targeted organ, e.g., brain, heart, kidney, skin, or lung. Similarly dosages are adjusted for administration to other organs.

Provided herein are methods to treat tuberous sclerosis complex in a patient. In some embodiments, the invention provides a method to treat a renal angiomyolipoma, a LAM, or a brain dysfunction a subject. For each of the described methods, the treatment may be used to prevent the occurrence of further damage or to rescue tissue having mild or advanced disease. As used herein, the term “rescue” means to prevent progression of the disease, prevent spread of damage to uninjured cells or to improve damage in injured cells.

Thus, in one embodiment, the composition is administered before disease onset. In another embodiment, the composition is administered prior to the development of symptoms. In another embodiment, the composition is administered after development of symptoms. In yet another embodiment, the composition is administered when less than 90% of the target cells are functioning or remaining, e.g., as compared to a reference tissue. In yet another embodiment, the composition is administered when more than 10% of the target cells are functioning or remaining, e.g., as compared to a reference tissue. In yet another embodiment, the composition is administered when more than 20% of the target cells are functioning or remaining. In yet another embodiment, the composition is administered when more than 30% of the target cells are functioning or remaining.

In yet another embodiment, any of the above described methods is performed in combination with another, or secondary, therapy. The therapy may be any now known, or as yet unknown, therapy which helps prevent, arrest or ameliorate these mutations or defects or any of the effects associated therewith. The secondary therapy can be administered before, concurrently with, or after administration of the cTuberin nucleic acid molecule or rAAV carrying a cTuberin nucleic acid molecule as described above. In one embodiment, a secondary therapy involves the treatment of seizures in the subject, including, for example, the administration of an anti-seizure drug. In a further embodiment, the secondary therapy involves the administration of rapamycin. In a further embodiment, the secondary therapy involves co-administration with rapamycin. The administration or co-administration of rapamycin can be to a subject having tuberous sclerosis complex. Further, the administration or co-administration of rapamycin can be to a subject having a renal angiomyolipoma, a LAM, or a brain dysfunction. In some embodiments, the administration or co-administration of rapamycin can be during early childhood at the time of infantile seizures. In further embodiments, the administration or co-administration of rapamycin can be after detection of subependymal overgrowths by, for example, MRI. In further embodiments, the administration or co-administration of rapamycin can be any time later in life due to symptoms caused by overgrowths due to somatic loss of tuberin function.

For use in these methods, the volume and viral titer of each injection is determined individually. The dosages, administrations, and regimens may be determined by the attending physician given the teachings of this disclosure.

### EXAMPLES

The following are examples of the methods and compositions of the invention. It is understood that various other embodiments may be practiced, given the general description provided above.

### Example 1. Cell Culture

Tsc2-null mouse embryonic fibroblasts (MEFs) (Huang et al., Biochem. J. 412(2):179-190 2008) and immortalized TR1102 human angiomyolipoma cells (Hong et al., Mol. Cell. 30:701-711, 2008; Yu et al., Am. J. Physiol. Lung Cell. Mol. Physiol. 286:L694-1700, 2004) were grown in Dulbecco's Modified Eagle's medium (DMEM) (Cellgro®, Manassas, VA) growth media, supplemented with 10% fetal bovine serum (FBS) (Sigma-Aldrich®, St. Louis, MO) and 1% penicillin/streptomycin (Cellgro®) and cells were maintained at 37° C. in a humidified atmosphere of 5% C02 and 95% air. Lentivirus vectors were generated using CSCW-IG, a self-inactivating lentiviral vector, which has a CMV promoter controlling expression of both transgene and GFP cDNAs separated by an IRES element (Sena-Esteves et al., J. Virol. Methods. 122(2):131-139, 2004). The cDNA-encoding Fluc (pGL3-basic; Promega®, Madison, WI) and monomeric red fluorescent protein (mCherry) (Rizzo et al., 2004) were amplified by PCR. Fluc sequences were inserted directly downstream of the CMV promoter at the Nhe I site and mCherry sequences were inserted in place of the GFP cDNA at Bsa I and Sal I sites, generating pCSCW-Fluc-IRES-mCherry. Lentivirus vectors were generated as described with a typical titer of 10⁸-10¹⁰ transducing units (tu) per ml (Sena-Esteves et al., J. Virol. Methods. 122(2):131-139, 2004). To confer stable expression of Fluc and mCherry on lymphangioleiomyomatosis (LAM) cells, they were infected with CSCW-Fluc-IRES-mCherry lentivirus at a multiplicity of infection (M.O.I) of 100, which gave >90% infectability (cell line termed as TSC2-LAM-FC). COS-7 cells were cultured in DMEM supplemented with 10% FBS and 1% penicillin/streptomycin and transfected with a plasmid vector containing cDNA for cTuberin (pAAV-CBA-cTuberin) using Lipofectamine™ 3000 (Thermo Fisher Scientific®, Waltham, MA).

### Example 2. AAV Vector Design and Packaging

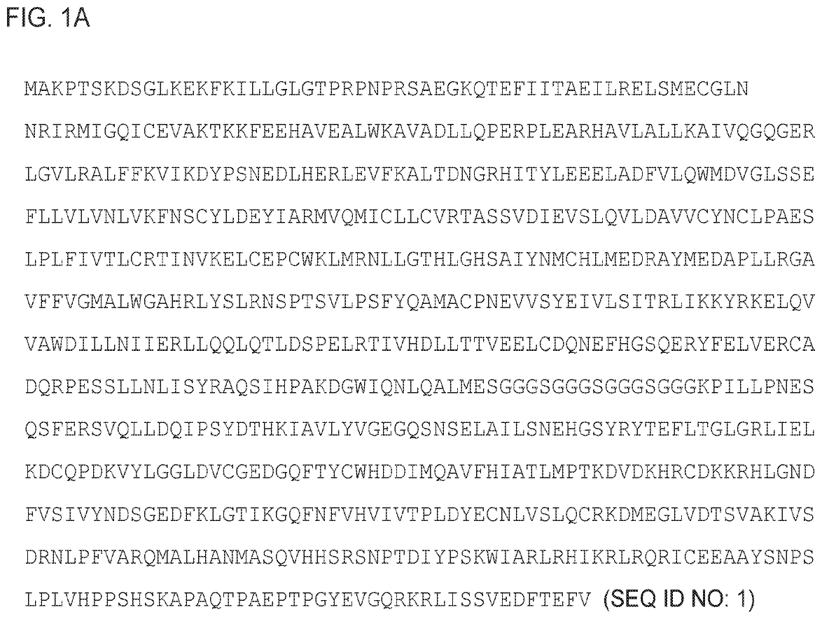

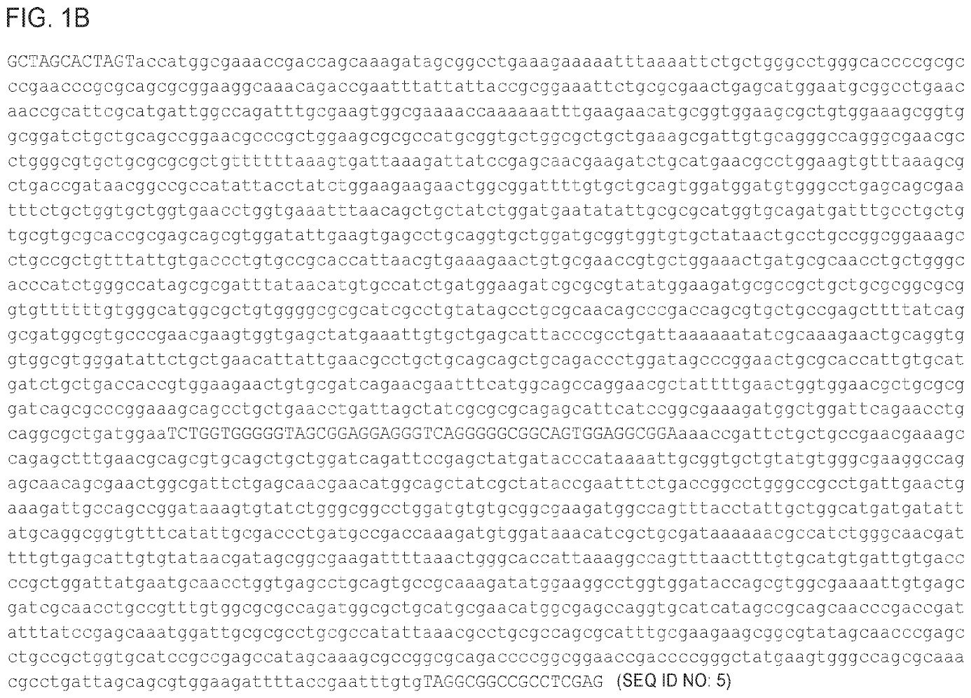

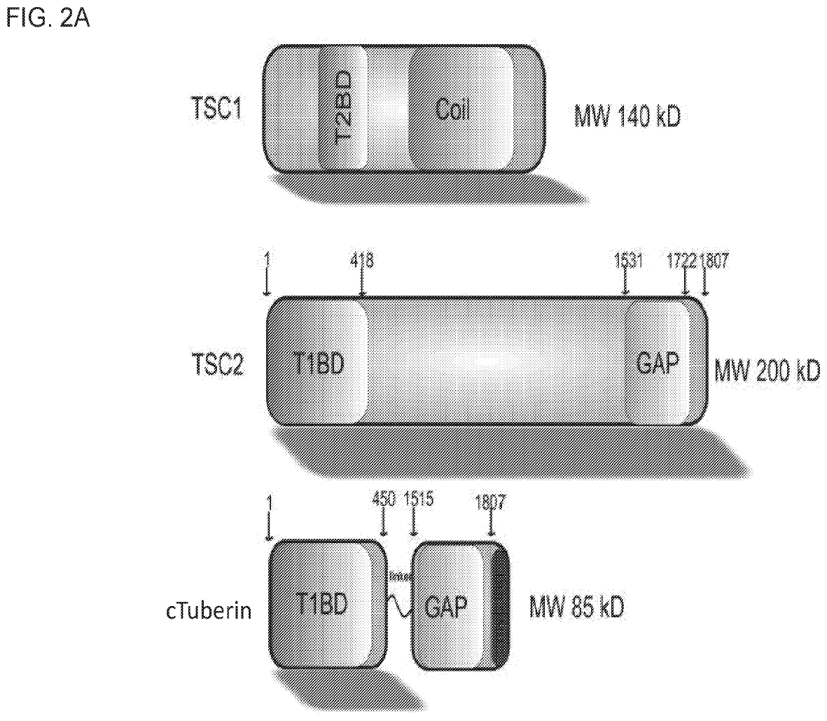

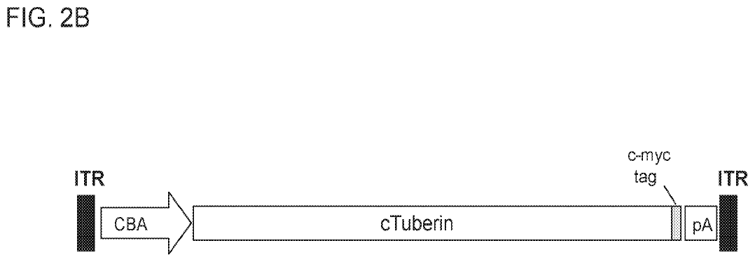

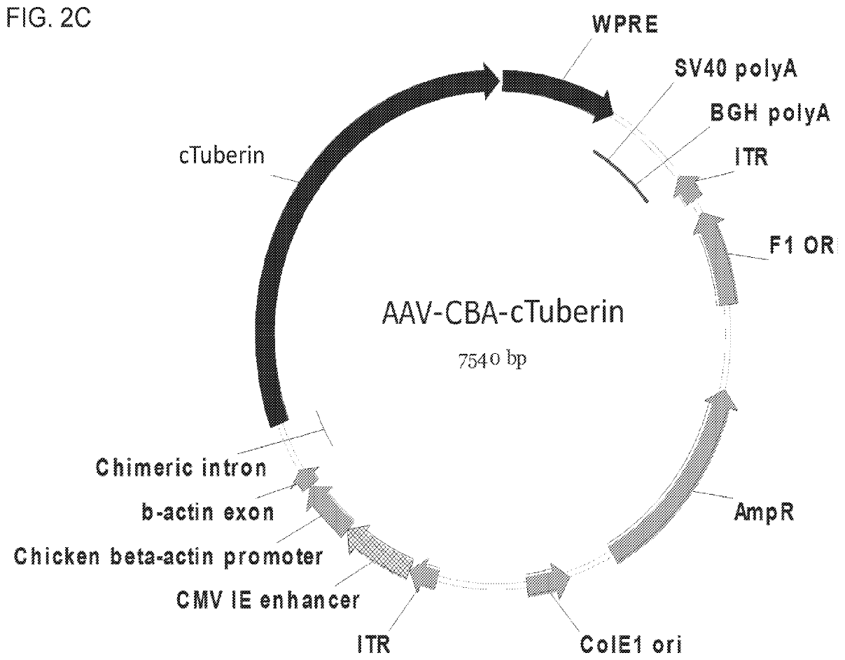

The AAV vector plasmid AAV-CBA-Cre-BGHpA was derived as described in Prabhakar et al., PLoS One. 8(5):e64224, 2013. These AAV vectors carry AAV2 ITR elements and gene expression is controlled by a hybrid promoter (chicken β-actin (CBA)) composed of the CMV immediate/early gene enhancer fused to the beta-actin promoter (Gray et al., Hum. Gene Ther. 22:1143-1153 2011). The AAV vector plasmid AAV-CBA-cTuberin was derived from the plasmid pAAV-CBA-W (CSCW-IG) (Sena-Esteves et al., J. Virol. Methods. 122(2):131-139, 2004). This vector contains the CBA promoter driving cTuberin, followed by a wood chuck hepatitis virus posttranscriptional regulatory element (WPRE) and SV40 and bovine growth hormone (BGH) polyadenylation (poly A) signal sequences (FIG. 2C). The condensed tuberin (cTuberin) construct contains: ACC (Kozak sequence)::amino acids 1-450 of human tuberin::gly/ser linker::amino acids 1515-1807 of human tuberin::cmyc tag. The 2,307 bp cDNA sequence encodes an 85 kDa protein (FIG. 1A).

AAV1 and AAV9 serotype vectors were produced by transient co-transfection of 293T cells by calcium phosphate precipitation of vector plasmids (AAV-CBA-cTuberin-cmyc), adenoviral helper plasmid pFΔ6, and a plasmid encoding AAV9 (pXR9) or AAV1 (pXR1) capsid genes, as previously described in Broekman et al., Neuroscience. 138:501-510, 2006. The identity of all PCR amplified sequences was confirmed by sequencing. Briefly, AAV vectors were purified by iodixanol density gradient centrifugation. The virus-containing fractions were concentrated using Amicon® Ultra 100 kDa MWCO centrifugal devices (EMD Millipore®, Billerica, MA) and the titer (genome copies (gc)/ml) was determined by real-time PCR amplification with primers and probe specific for the bovine growth hormone polyadenylation signal.

### Example 3. Western Blots

Briefly, cultured cells were lysed in lysis buffer (50 mM HEPES pH 8.0, 150 mM NaCl, 2 mM EDTA, 2.5% sodium dodecyl sulfate, 2% CHAPS, 2.5 mM sucrose, 10% glycerol, 10 mM sodium fluoride, 2 mM sodium vanadate, 1 mM PMSF, 10 mM sodium pyrophosphate, protease inhibitor cocktail). After sonication and incubation at 8° C. for 10 min, the samples were centrifuged at 14,000 g for 30 min at 8° C. Equal amounts of protein, determined by detergent-compatible protein assay kit (Bio-Rad®, Hercules, CA), were boiled for 5 min in Laemmli sample buffer, separated by SDS-PAGE, and transferred onto nitrocellulose membranes (Bio-Rad®). The equal protein loading was confirmed by Ponceau S staining. The membranes were blocked in 2% blocking reagent (GE Healthcare, Pittsburgh, PA) for 1 h at room temperature and incubated with primary antibodies overnight at 4° C. Anti-Tuberin/TSC2 (#3612), anti-phospho-S6 (#2211), anti-S6 (#2212) (Cell Signaling Technology®), and anti-glyceraldehyde-3-phosphate dehydrogenase (GAPDH) (#2275-PC) (Trevigen®, Gaithersburg, MD) antibodies were used as primary antibodies. Anti-rabbit or anti-mouse IgG antibody conjugated with horseradish peroxidase was used as a secondary antibody. Enhanced chemiluminescence reagent, Lumigen® ECL Ultra (TMA-6) (Lumigen®, Southfield, MI) was used to detect the antigen-antibody complex.

### Example 4. Animals and Intracerebroventricular (ICV) Injections

Experimental research protocols were approved by the Institutional Animal Care and Use Committee (IACUC) for the Massachusetts General Hospital (MGH) following the guidelines of the National Institutes of Health for the Care and Use of Laboratory Animals. Experiments were performed on Tsc2^{c/c} floxed mice (Onda et al., J. Clin. Invest. 104(6):687-695, 1999). In response to Cre recombinase, the Tsc2^{c/c} alleles are converted to null alleles, and the lacZ allele expresses β-galactosidase. These mice have a normal, healthy lifespan.

For vector injections, on the day of birth (postnatal day 0 (P0)) or on P3, neonates were cryo-anesthetized and injected with 1 μl or 2 μl of viral vector AAV1-CBA-Cre into each cerebral lateral ventricle with a glass micropipette (70-100 mm diameter at the tip) using a Narishige® IM300 microinjector at a rate of 2.4 psi/see (Narshige International, East Meadow, NY). Mice were then placed on a warming pad and returned to their mothers after regaining normal color and full activity typical of newborn mice.

### Example 5. Retro-Orbital (RO) Injections

At 3 weeks of age (P21) mice were anesthetized by isoflurane inhalation (3.5% isoflurane in an induction chamber, then maintained anesthetized with 2-3% isoflurane and 1-2 liter/min oxygen for the duration of the experiment). AAV vectors were injected retro-orbitally into the vasculature right behind one of the eyeballs in a volume of 70 μl of solution (10 μl of AAV1- or AAV9-CBA-cTuberin+60 μl saline) or non-injected using a 0.3 ml insulin syringe over less than 2 min (Yardeni et al., Lab. Anim. (NY). 40(5):155-160, 2011).

### Example 6. Subcutaneous Lymphangioleiomyomatosis (LAM) Model

Three million human TSC2 null, immortalized angiomyolipoma cells expressing Fluc were suspended in 50 μl reduced serum media (Opti-MEM®, Gibco®), mixed with 50 μl of Matrigel® (BD Matrigel™ Matrix HC) (BD Biosciences, Bedford, MA) and implanted subcutaneously in the backs of NOD-SCID II2R gamma (NSG™) mice. After 4 weeks, mice were injected intraperitoneally with the Fluc substrate D-luciferin (LUCNA-1G) (Gold Biotechnology®, St. Louis, MO), and a signal was acquired 5 min later with a high efficiency IVIS® Spectrum (Caliper Life Sciences, Hopkinton, MA) with an XGI-8 gas anesthesia system (Caliper Life Sciences).

### Example 7. Histology and Immunohistochemistry (IHC)

Standard histology of mouse brains was carried out as described in Prabhakar et al., PLoS One. 8(5):e64224, 2013. Five μm sections were stained with Haematoxylin and Eosin (H&E) or used for IHC, as described (ibid.) using antibodies for pS6 (#2211, Cell Signaling), with secondary antibodies, as described (ibid.).

### Example 8. Statistical Analysis

All analyses of survival curves (chi-squared test) were performed using GraphPad Prism software (GraphPad Software, Inc., La Jolla, CA). The P-values depicted are statistically significant.

### Example 9. Expression of cTuberin in COS-7 Cells Transfected with AAV-CBA-cTuberin Vector Plasmid

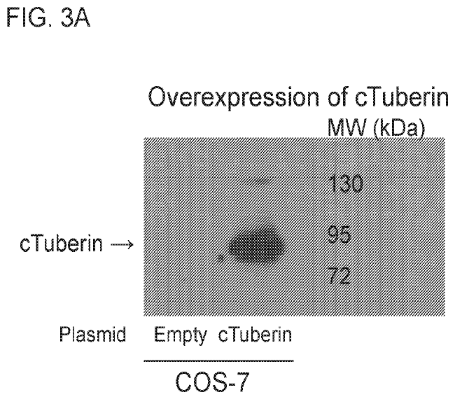

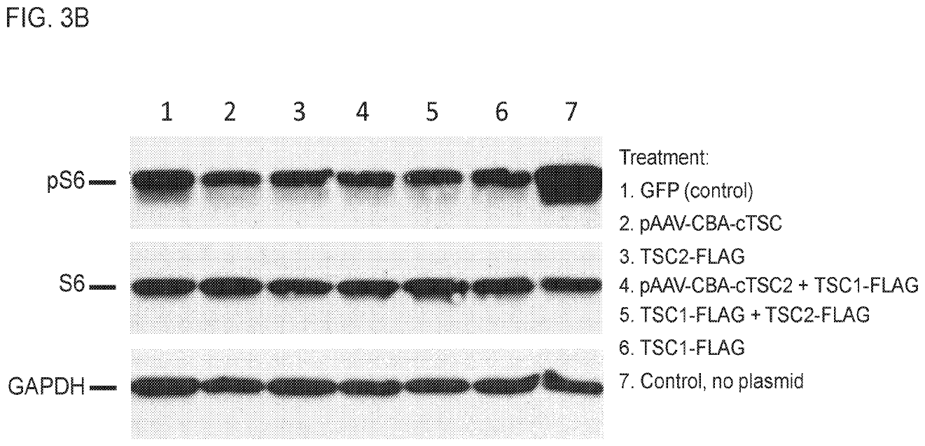

COS-7 cells were transfected according to the procedure in Example 1 with the AAV-CBA-cTuberin vector plasmid of Example 2. After 24 hours, expression of cTuberin was detected by Western blotting as described in the preceding examples with anti-Tuberin/TSC2 antibody. Expression of cTuberin was apparent at the expected molecular weight (MW) of 85 kDa, as shown in FIG. 3A.

### Example 10. pS6 Kinase Activity in COS-7 Cells Transfected with Various AAV Constructs

To test the activity of cTuberin, COS-7 cells were transfected with GFP, pAAV-CBA-cTSC2, TSC2-FLAG, pAAV-CBA-cTSC2+TSC1-FLAG, TSC1-FLAG+TSC2-FLAG, and TSC1-FLAG vectors. Expression levels of phosphorylated S6 (pS6), S6, and GAPDH were detected by Western blotting. While pS6 kinase levels are normally elevated in the absence of tuberin activity, cells transfected with the AAV-CBA-cTuberin plasmid showed lower pS6 levels, indicating decreased pS6 kinase activity. This is shown in FIG. 3B, where pS6 levels are elevated in columns 1 (GFP (control)) and 7 (control, no plasmid) as compared to the other cells.

### Example 11. Survival of Mice Injected P0 with AAV1-CBA-Cre Vector, Mice Injected P21 with AAV9-CBA-cTuberin Vector, and Non-Injected Mice

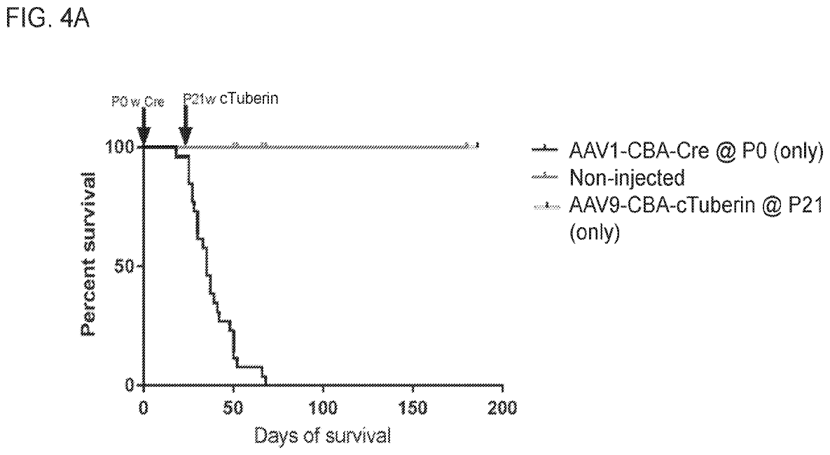

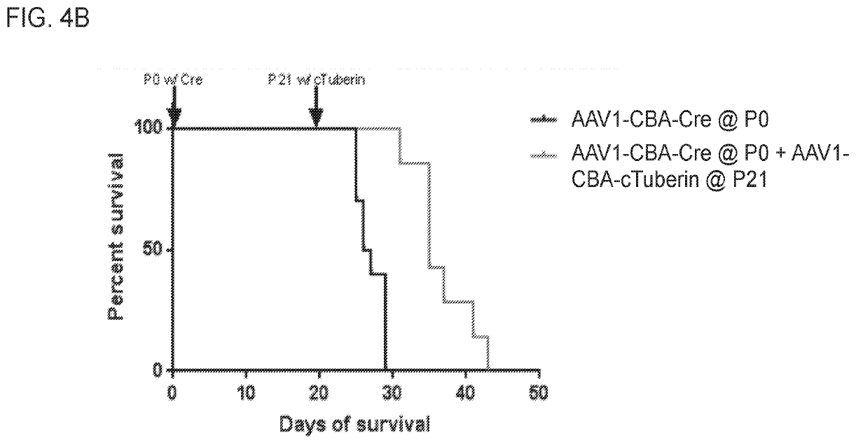

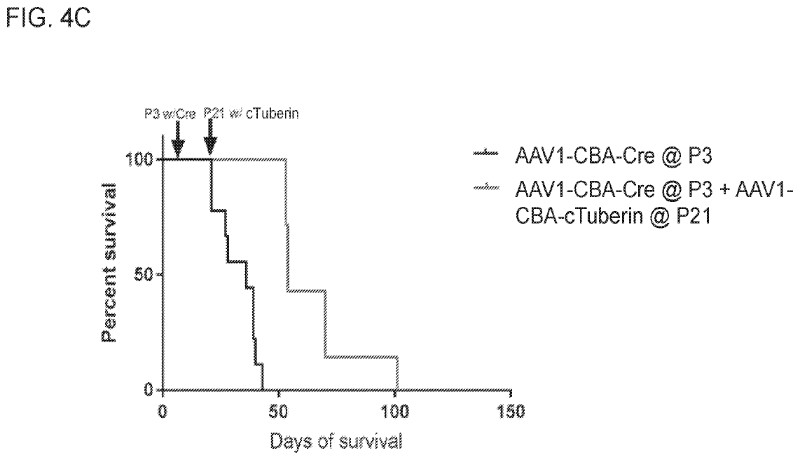

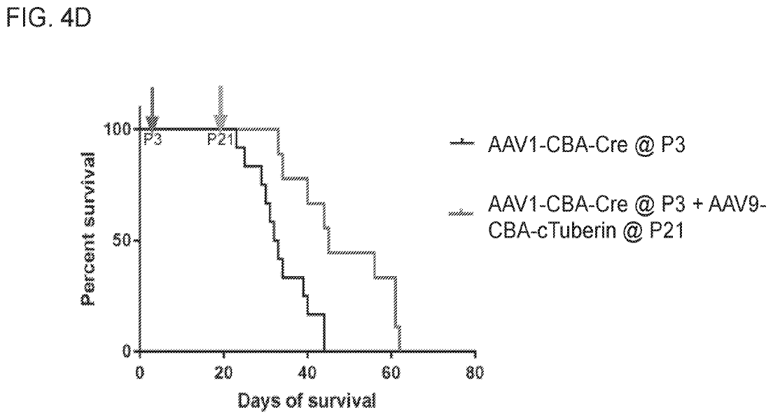

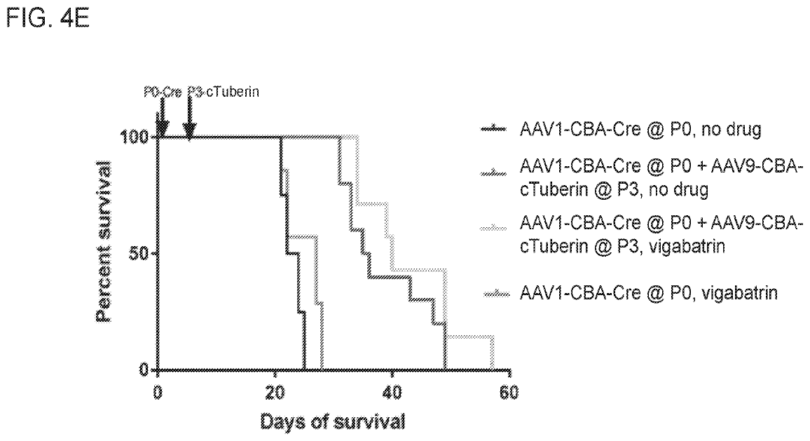

The efficacy of the AAV-CBA-cTuberin vector was tested on Tsc2^{c/c} mice. Intracereboventricular (ICV) and retro-orbital (RO) injections were carried out as described in the preceding examples. AAV1-CBA-Cre and AAV9-CBA-cTuberin vectors were prepared as described above. Mice were injected ICV at P0 with AAV1-CBA-Cre (N=16), injected RO at P21 with AAV9-CBA-cTuberin (N=7), or non-injected (N=6). The titers of the AAV1-CBA-Cre and AAV9-CBA-cTuberin injections were 9.1×10¹² g.c./ml and 4.5×10¹² g.c./ml, respectively. The median survival was 35 days for the AAV1-CBA-Cre mice, >175 days for the non-injected mice, and >185 days for the AAV9-CBA-cTuberin mice. The difference between the groups was P<0.0001 (log-rank) or P<0.0001 (Gehan-Breslow-Wilcoxon), both statistically significant. Survival curves are depicted in FIG. 4A.

### Example 12. Survival and Histology of Mice Injected P0 with AAV1-CBA-Cre Vector+P21 with AAV1-CBA-cTuberin Vector

The efficacy of gene therapy with the AAV1-CBA-cTuberin vector was tested on mice lacking tuberin. The AAV1-CBA-cTuberin vector was prepared as described in the preceding examples. All mice were injected ICV at P0 with AAV1-CBA-Cre. At P21, one group of mice was injected RO with AAV1-CBA-cTuberin (N=7), while a second group was non-injected (N=10). The titers of the AAV1-CBA-Cre and AAV1-CBA-cTuberin injections were 5.1×10¹³ g.c./ml and 3×10¹¹ g.c./ml, respectively. The median survival of the non-injected mice was 26.5 days, while the AAV1-CBA-cTuberin injected mice survived for a median of 35 days. The difference between the two groups was P=0.0001 (log-rank) or P=0.0004 (Gehan-Breslow-Wilcoxon), both statistically significant. Survival curves are depicted in FIG. 4B.

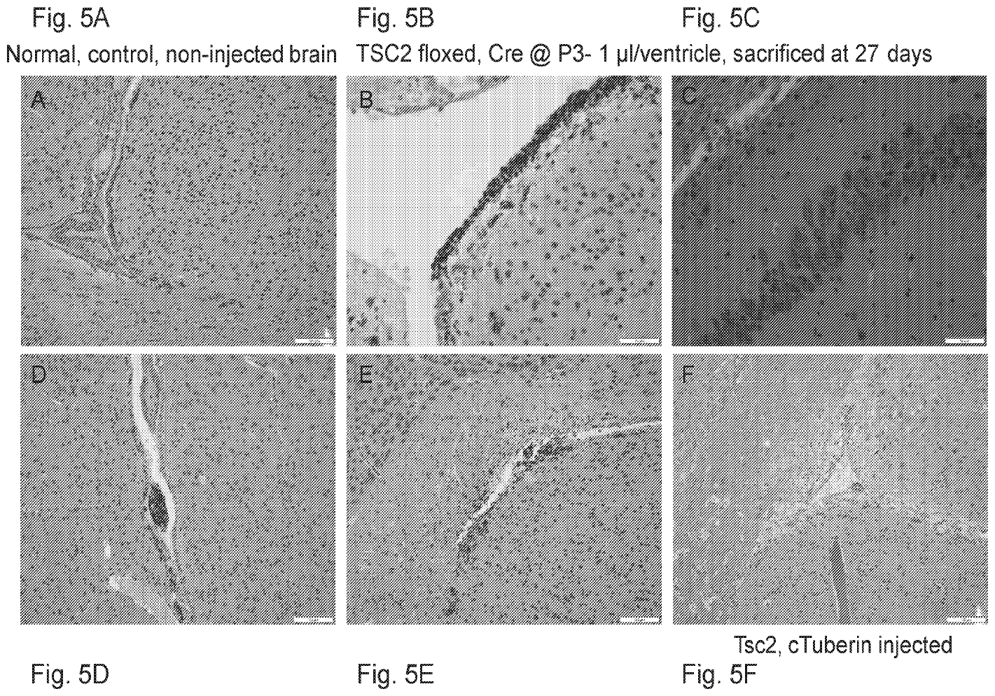

Additionally, the brains of the tuberin-lacking mice and AAV-CBA-cTuberin treated mice were studied using H&E staining or IHC for pS6 performed as described in Example 7. Tsc2^{c/c} mice were injected at P0 with AAV1-CBA-Cre and at P21 AAV1-CBA-cTuberin according to the same design of the preceding experiment, then sacrificed at P27. Results are shown in FIGS. 5A-5F. FIG. 5A shows the staining in a normal, non-injected brain (control). FIGS. 5B-5E show the staining in a mouse injected only with AAV1-CBA-Cre at P0, showing proliferation of ependymal cells (FIG. 5B), enlarged pyramidal cell in the hippocampus (FIG. 5C), a subependymal nodule (FIG. 5D), and multiple subependymal nodules and proliferation (FIG. 5E). Finally, in the mouse treated with AAV1-CBA-cTuberin at P21, FIG. 5F shows a very small nodule, inflammation, and edema in the subependymal region.

### Example 13. Survival of Mice Injected P3 with AAV1-CBA-Cre Vector+P21 with AAV1- or AAV9-CBA-cTuberin Vectors

The efficacy of gene therapy with either AAV1-CBA-cTuberin vector or AAV9-CBA-cTuberin vector was further tested in the following two experiments. Mice were initially injected at P3 instead of P0 with AAV1-CBA-Cre when the cerebral spinal fluid (CSF) barrier is somewhat less penetrable than at P0, which should cause less loss of tuberin in the brain.

### AAV1-CBA-cTuberin

All Tsc2^{c/c} mice were injected ICV at P3 with AAV1-CBA-Cre. At P21, one group of mice was injected RO with AAV1-CBA-cTuberin (N=9), while a second group was non-injected (N=7). The titers of the AAV1-CBA-Cre and AAV1-CBA-cTuberin injections were 5.1×10¹³ g.c./ml and 3×10¹¹ g.c./ml, respectively. The median survival of the non-injected mice was 36 days, while the AAV1-CBA-cTuberin injected mice survived for a median of 54 days. The difference between the two groups was P<0.0001 (log-rank) or P=0.0004 (Gehan-Breslow-Wilcoxon), both statistically significant. Survival curves are depicted in FIG. 4C.

### AA V9-CBA-cTuberin

All Tsc2^{c/c} mice were injected ICV at P3 with AAV1-CBA-Cre. At P21, one group of mice was injected RO with AAV9-CBA-cTuberin (N=11), while a second group was non-injected (N=9). The titers of the AAV1-CBA-Cre and AAV9-CBA-cTuberin injections were 5.1×10¹³ g.c./ml and 4.5×10¹² g.c./ml, respectively. The median survival of the non-injected mice was 32 days, while the AAV9-CBA-cTuberin injected mice survived for a median of 45 days. The difference between the two groups was P<0.0006 (log-rank) or P=0.0014 (Gehan-Breslow-Wilcoxon), both statistically significant. Survival curves are depicted in FIG. 4D.

### Example 14. Survival of Mice Injected with AAV9-CBA-cTuberin Vector and Vigabatrin

It is possible that the therapeutic vector will also decrease seizures. If not, seizures may be the cause of early death, i.e., death prior to hydrocephalus produced by subependymal nodules (SENs). The efficacy of AAV9-CBA-cTuberin was further tested in combination with vigabatrin, which is effective at blocking seizures in Tsc1-floxed/GFAP-Cre mice (Zhang et al., PLoS One. 8(2):e57445, 2013).

All mice were injected ICV at P0 with AAV1-CBA-Cre. One group of mice was injected RO at P3 with AAV9-CBA-cTuberin, while a second group was non-injected. Of the injected mice, one group was treated with vigabatrin (50 mg/kg) (N=7), while a second group was untreated (N=10). Of the non-injected mice, one group was treated with vigabatrin (200 mg/kg) (N=7), while a second group was untreated (N=8). The titers of the AAV1-CBA-Cre and AAV9-CBA-cTuberin were 5.1×10¹³ g.c./ml and 4.5×10¹² g.c./ml, respectively. The dose of vigabatrin delivered was 50 mg/kg for the AAV9-CBA-cTuberin injected mice and 200 mg/kg for the non-injected mice. The median survival was 23 days for the non-injected, no drug mice; 27 days for the non-injected, vigabatrin-treated mice; 35.5 days for the injected, no drug mice; and 40 days for the injected, vigabatrin-treated mice. The difference between the two AAV9-CBA-cTuberin injected groups was P<0.0001 (log-rank) or P<0.0001 (Gehan-Breslow-Wilcoxon), both statistically significant.

### Example 15. Efficacy of AAV9-CBA-cTuberin on LAM Tumors In Vivo

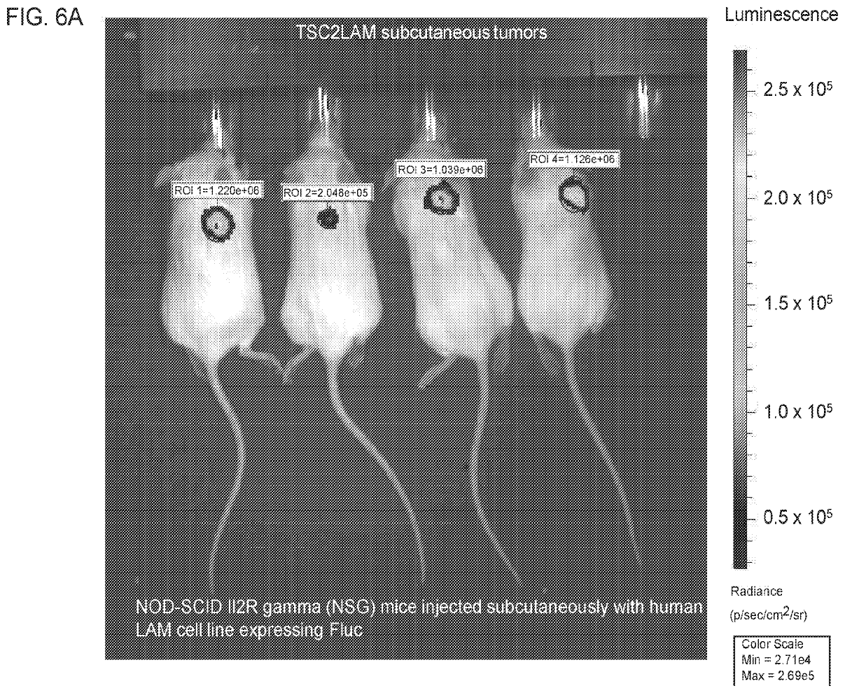

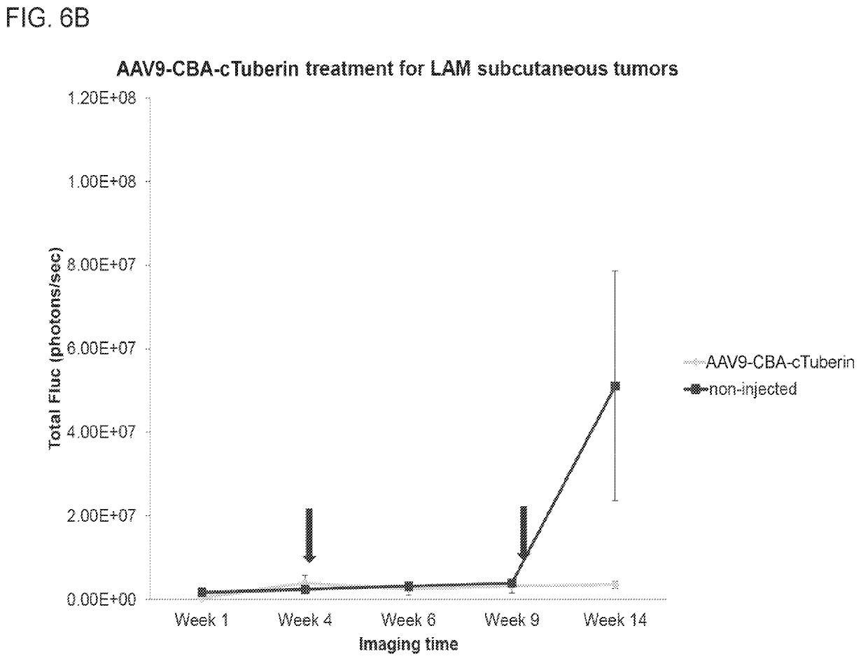

The efficacy of AAV9-CBA-cTuberin was also tested in vivo on lymphangioleiomyomatosis (LAM) tumors injected subcutaneously in immunocompromised NSG mice, which were prepared as described in Example 4. The Fluc-expressing LAM tumors are shown in FIG. 6A. Tumor volume was monitored via bioluminescence at weeks 1, 4, 6, 9 and 14. At weeks 4 and 9, tumors were either injected with AAV9-CBA-cTuberin vector (N=7) or non-injected (N=5). The titer of the AAV9-CBA-cTuberin vector was 4.3×10¹⁰ g.c./ml. By week 14, tumors injected with the cTuberin vector had ceased increasing in size, while the non-injected tumors continued to expand in volume, as depicted in FIG. 6B.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An engineered condensed tuberin (cTuberin) comprising a hamartin binding region and a GTPase-activating protein (GAP) region, but lacking an Akt phosphorylation site Thr 1462.

2. The cTuberin of claim 1, wherein said hamartin binding region has at least 90% sequence identity to SEQ ID NO: 2.

3. The cTuberin of claim 2, wherein said hamartin binding region is SEQ ID NO: 2.

4. The cTuberin of claim 1, wherein said GAP region has at least 90% sequence identity to SEQ ID NO: 3.

5. The cTuberin of claim 4, wherein said GAP region is SEQ ID NO: 3.

6. The cTuberin of claim 1, wherein said cTuberin lacks amino acids 451-1514 of human tuberin (SEQ ID NO: 10).

7. The cTuberin of claim 1, wherein said cTuberin comprises a spacer between said hamartin binding region and GAP region.

8. The cTuberin of claim 1, wherein said spacer comprises at least SGGG.

9. (canceled)

10. The cTuberin of claim 1, wherein said cTuberin has at least 90% sequence identity to SEQ ID NO: 1.

11. (canceled)

12. A nucleic acid molecule encoding the cTuberin of claim 1.

13. The nucleic acid molecule of claim 12, wherein said nucleic acid molecule is codon optimized for expression in a human cell.

14. The nucleic acid molecule of claim 13, wherein said nucleic acid molecule is operably linked to a regulatory control sequence.

15-17. (canceled)

18. The nucleic acid molecule of claim 12, wherein said nucleic acid molecule has at least 90% sequence identity to SEQ ID NO: 5.

19-20. (canceled)

21. A cell or virus comprising the nucleic acid molecule of claim 12.

22. A composition comprising the nucleic acid molecule of claim 12.

23. A recombinant adeno-associated virus (rAAV), said rAAV comprising an AAV capsid and an AAV genome packaged therein, said AAV genome comprising a nucleic acid molecule capable of expressing an engineered cTuberin comprising a hamartin binding region and a GAP region, but lacking an Akt phosphorylation site Thr 1462.

24-29. (canceled)

30. A composition comprising the rAAV of claim 23 and a pharmaceutically acceptable carrier.

31. A method of treating a patient having tuberous sclerosis complex (TSC), said method comprising administering to said patient an engineered cTuberin comprising a hamartin binding region and a GAP region, but lacking an Akt phosphorylation site Thr 1462.

32-34. (canceled)

35. The method of claim 31, wherein said patient has a renal angiomyolipoma.

36-47. (canceled)

48. The method of claim 31, wherein said patient is further administered rapamycin.
